(19) United States
(12) Patent Application Publication
(10) Pub. No.: US 2026/0022169 A1
(43) Pub. Date: Jan. 22, 2026

(54) MODIFIED ANTIBODIES AND USES THEREOF

(71) Applicants: ELPISCIENCE BIOPHARMA, LTD., Shanghai (CN); ELPISCIENCE (SUZHOU) BIOPHARMA, LTD., Suzhou, Jiangsu (CN)

(72) Inventors: Shanshan WANG, Shanghai (CN); Jinfeng ZHAO, Shanghai (CN); Xiang XU, Shanghai (CN); Zhihao WU, Shanghai (CN); Dawei SUN, Shanghai (CN); Hongtao LU, Shanghai (CN)

(21) Appl. No.: 18/845,875

(22) PCT Filed: Mar. 10, 2023
(86) PCT No.: PCT/CN2023/080782

§ 371 Date: Sep. 10, 2024

(30) Foreign Application Priority Data
Mar. 11, 2022 (WO) PCT/CN2022/080398
Feb. 22, 2023 (WO) PCT/CN2023/077698

(51) Int. Cl. C07K 16/28

(52) U.S. Cl. CPC C07K 16/28; C07K 2317/24; C07K 2317/31; C07K 2317/35; C07K 2317/522; C07K 2317/526; C07K 2317/55; C07K 2317/624

(57) ABSTRACT

Provided are modified antibodies, the preparing method and the uses thereof. In particular, the modified antibodies are bi-specific antibodies or multi-specific antibodies. The heavy chains and light chains of the modified antibodies are paired with high assembly accuracy and stability.

### FIELD OF THE INVENTION

The present disclosure generally relates to modified antibodies and uses thereof.

### BACKGROUND

Currently, monoclonal antibodies have been used as specific and effective biological drugs, which can display exquisite specificity for a single target antigen. However, for some situations, single antibodies with specificity for two (or more) different target antigens-often called bispecific antibodies (or multi-specific antibodies)—are preferred. Bispecific or multi-specific antibodies can simultaneously recognize two or more different antigens, neutralize different pathogenic mediators, recruit different type of effector cells, and modulate signal pathways, which makes them superior to monospecific antibodies in many aspects. Under such a circumstance, the development of bispecific or multi-specific antibodies as therapeutic agents for human diseases has great clinical significance and bispecific antibodies have become widely used formats in recent years for diagnostic and therapeutic applications.

However, production of bispecific or multi-specific antibodies has been challenging. In the case of a bispecific antibody, the heavy chain specific for one antigen must pair with the light chain specific for the same antigen. If a heavy chain pairs with the light chain specific for the other different antigen, the intended antigen specificities may be destroyed or reduced. Since it's still not possible to well control the correct pairing between a light chain with the corresponding heavy chain possessing a native IgG structure, efforts to introduce additional antibody specificities into a single antibody often result in the production of misassembled species.

Needs remain for production of novel modified antibodies with more than one specificities.

### SUMMARY OF THE INVENTION

In one respect, the present disclosure provides an antibody or antigen-binding fragment thereof, which comprises a first arm formed by a first heavy chain and a first light chain with an interchain disulfide bond formed therebetween, and a second arm formed by a second heavy chain and a second light chain with an interchain disulfide bond formed therebetween, wherein at least one non-cysteine residue on the first heavy chain is substituted with cysteine, wherein the non-cysteine residue is at a position selected from the group consisting of amino acid positions 126, 128, 129, 136, 141, 168, 170, 173, 175 or 187 of the first heavy chain; and at least one non-cysteine residue on the first light chain is substituted with cysteine, wherein the non-cysteine residue is at a position selected from the group consisting of amino acid positions 114, 116, 118, 124, 135, 137, 138, 160, 162 or 164 of the first light chain.

In some embodiments, the at least one non-cysteine residue on the first heavy chain substituted with cysteine is selected from the group consisting of amino acid residues F126, L128, A129, S136, A141, H168, F170, V173, Q175 or T187 of the first heavy chain; and the at least one non-cysteine residue on the first light chain substituted with cysteine is selected from the group consisting of amino acid residues S114, F116, F118, Q124, L135, N137, N138, Q160, S162 or T164 of the first light chain.

In some embodiments, the first light chain and the first heavy chain comprises at least one non-cysteine substitution pair of a native cysteine on the first light chain and a native cysteine on the first heavy chain, and at least one cysteine substitution pair selected from the group consisting of: (i) S114C on light chain and S136C on heavy chain; (ii) F116C on light chain and S136C on heavy chain; (iii) F118C on light chain and L128C on heavy chain; (iv) F118C on light chain and A129C on heavy chain; (v) F118C on light chain and A141C on heavy chain; (vi) Q124C on light chain and F126C on heavy chain; (vii) L135C on light chain and F170C on heavy chain; (viii) N137C on light chain and F170C on heavy chain; (ix) N138C on light chain and H168C on heavy chain; (x) Q160C on light chain and V173C on heavy chain; (xi) Q160C on light chain and Q175C on heavy chain; (xii) S162C on light chain and F170C on heavy chain; (xiii) S162C on the light chain and T187C on heavy chain; and (xiv) T164C on light chain and F170C on heavy chain; wherein the at least one cysteine substitution pair forms at least one interchain disulfide bond between the first heavy chain and the first light chain; wherein the native cysteines on the first heavy chain and the first light chain are independently substituted with any one of non-cysteine residues.

In some embodiments, the native cysteines on the first heavy chain and the first light chain are independently substituted with any one of Serine, Alaine, Glycine or Valine. In some embodiments, the native cysteines to be substituted is amino acid reside C214 in the light chain and amino acid residue C220 in the heavy chain of the antibody. In some embodiments, the first light chain comprises mutation C214S and the first heavy chain comprises mutation C220S.

In some embodiments, the first heavy chain and the first light chain comprises one cysteine substitution pair. In some embodiments, the first heavy chain and the first light chain comprises two cysteine substitution pairs.

In some embodiments, the two cysteine substitution pairs are selected from the group consisting of: (i) S114C/Q160C on light chain and S136C/V173C on heavy chain; (ii) F118C/Q124C on light chain and L128C/F126C on heavy chain; (iii) F118C/Q124C on light chain and A129C/F126C on heavy chain; (iv) F118C/Q124C on light chain and A141C/F126C on heavy chain; (v) F116C/Q124C on light chain and S136C/F126C on heavy chain; (vi) Q124C/T164C on light chain and F126C/F170C on heavy chain; (vii) Q124C/N138C on light chain and F126C/H168C on heavy chain; (viii) Q160C/L135C on light chain and Q175C/F170C on heavy chain; (ix) S114C/S162C on light chain and S136C/F170C on heavy chain; (x) F118C/S162C on light chain and L128C/F170C on heavy chain; (xi) F118C/S162C on light chain and A141C/F170C on heavy chain; (xii) Q124C/S162C on light chain and F126C/F170C on heavy chain; and (xiii) N137C/S162C on light chain and F170C/T187C on heavy chain.

In some embodiments, the first light chain comprises mutations Q124C/T164C/C214S and the first heavy chain comprises mutations F126C/F170C/C220S. In some embodiments, the first light chain comprises mutations Q124C/S162C/C214S and the first heavy chain comprises mutations F126C/F170C/C220S.

In some embodiments, the disulfide bond between the second heavy chain and the second light chain is formed between a native cysteine on the second light chain and a native cysteine on the second heavy chain; wherein the position of the native cysteine on the second light chain corresponds to position C214 on the human kappa chain, and the position of the native cysteine on the second heavy chain corresponds to position C220 on the human IgG1.

In some embodiments, the second light chain and the second heavy chain comprises at least one non-cysteine substitution pair of a native cysteine substituted to Serine on the light chain and a native cysteine substituted to Serine on the heavy chain, and at least one cysteine substitution pair selected from the group consisting of: (i) S114C on light chain and S136C on heavy chain; (ii) F116C on light chain and S136C on heavy chain; (iii) F118C on light chain and L128C on heavy chain; (iv) F118C on light chain and A129C on heavy chain; (v) F118C on light chain and A141C on heavy chain; (vi) Q124C on light chain and F126C on heavy chain; (vii) L135C on light chain and F170C on heavy chain; (viii) N137C on light chain and F170C on heavy chain; (ix) N138C on light chain and H168C on heavy chain; (x) Q160C on light chain and V173C on heavy chain; (xi) Q160C on light chain and Q175C on heavy chain; (xii) S162C on light chain and F170C on heavy chain; (xiii) S162C on the light chain and T187C on heavy chain; and (xiv) T164C on light chain and F170C on heavy chain; wherein the at least one cysteine substitution pair forms at least one interchain disulfide bond between the second light chain and the second heavy chain, and the cysteine substitution pair(s) on the first arm is different from the cysteine substitution pair(s) on the second arm. A non-cysteine substitution pair refers to a pair of native cysteines (with one on the light chain and one on the heavy chain) that have been both mutated to amino acids that are not cysteine. A cysteine substitution pair refers to a pair of native non-cysteine amino acids (with one on the light chain and one on the heavy chain) that have been both mutated to cysteines.

In some embodiments, the native cysteine substituted on the second light chain is at position C214 on the human kappa chain, and the native cysteine substituted on the second heavy chain is at position C220 on the human IgG1.

In some embodiments, the second heavy chain and the second light chain comprises two cysteine substitution pairs. In some embodiments, the two cysteine substitution pairs are selected from the group consisting of: (i) S114C/Q160C on light chain and S136C/V173C on heavy chain; (ii) F118C/Q124C on light chain and L128C/F126C on heavy chain; (iii) F118C/Q124C on light chain and A129C/F126C on heavy chain; (iv) F118C/Q124C on light chain and A141C/F126C on heavy chain; (v) F116C/Q124C on light chain and S136C/F126C on heavy chain; (vi) Q124C/T164C on light chain and F126C/F170C on heavy chain; (vii) Q124C/N138C on light chain and F126C/H168C on heavy chain; (viii) Q160C/L135C on light chain and Q175C/F170C on heavy chain; (ix) S114C/S162C on light chain and S136C/F170C on heavy chain; (x) F118C/S162C on light chain and L128C/F170C on heavy chain; (xi) F118C/S162C on light chain and A141C/F170C on heavy chain; (xii) Q124C/S162C on light chain and F126C/F170C on heavy chain; and (xiii) N137C/S162C on light chain and F170C/T187C on heavy chain; wherein the cysteine substitution pair(s) on the first arm is different from the cysteine substitution pairs on the second arm.

In some embodiments, the first heavy chain constant region and/or the second heavy chain constant region comprises a human IgG1, IgG2, IgG3 or IgG4; and the first light chain constant region and/or the second light chain constant region comprises a human kappa light chain or a human lambda light chain.

In some embodiments, the first heavy chain and the second heavy chain form a heterodimer; and the Fc region of the first heavy chain constant region and/or the Fc region of the second heavy chain constant region comprises one or more modifications facilitating the heterodimerization.

In some embodiments, the Fc region of the first heavy chain interacts with the Fc region of the second heavy chain through a Knob/Hole structure. In some embodiments, the Fc region of the first heavy chain comprises the Knob mutations, and the Fc region of the second heavy chain comprises the Hole mutations; or the Fc region of the first heavy chain comprises the Hole mutations, and the Fc region of the second heavy chain comprises the Knob mutations. In some embodiments, the Knob mutations comprise T366W, and the Hole mutations comprise T366S/L368A/Y407V.

In some embodiments, the modifications facilitating the heterodimerization comprise introduction of cysteine residues capable of forming a disulfide bond.

In some embodiments, the Fc region of the first heavy chain constant region and the Fc region of the second heavy chain constant region comprise modifications in the CH3 regions, respectively; wherein the modifications in the two CH3 regions are selected from the following:

| Modification(s) in one | Modification(s) in the other |
|---|---|
| CH3 region | CH3 region |
| T366S/L368A/Y407V | T366W |
| S354C/T366W | Y349C/T366S/L368A/Y407V |
| T366Y | Y407T |
| T366W | Y407A |
| T394W | F405A |
| T366Y/F405A | T394W/Y407T |
| T366W/F405W | T394S/Y407A |
| F405W | T394S |
| D399C | K392C |
| T366W/D399C | T366S/L368A/K392C/Y407V |
| T366W/K392C | T366S/L368A/D399C/Y407V |
| S354C/T366W | Y349C/T366S/L368A/Y407V |
| Y349C/T366W | S354C/T366S/L368A/Y407V |
| E356C/T366W | Y349C/T366S/L368A/Y407V |
| Y349C/T366W | E356C/T366S/L368A/Y407V |
| E357C/T366W | Y349C/T366S/L368A/Y407V |
| Y349C/T366W | E357C/T366S/L368A/Y407V |

In some embodiments, the Fc region of the first heavy chain constant region and the Fc region of the second heavy chain constant region comprise modifications in the CH3 regions, respectively; wherein the modifications in the two CH3 regions are selected from the following:

| Modification(s) in one | Modification(s) in the other |
|---|---|
| CH3 region | CH3 region |
| K370E/D399K/K439D | D356K/E357K/K409D |
| K409D | D399K |
| K409E | D399K |
| K409E | D399R |
| K409D | D399R |
| D339K | E356K |
| E356K/D399K | K392D/K409D |
| E356K/D399K | K409D/K439D |
| E357K/D399K | K370D/K409D |
| D399K/E357K/E356K | K370D/K392D/K409D |
| E357K/D399K | K392D/K409D |
| K392D/K409D | D399K |
| K360D/K409D | D399K |

In some embodiments, the first arm and the second arm can specifically bind to an antigen selected from the group consisting of: SIRPα, CLDN18.2, Siglec15, HER2, EGFR, CD19, CD20, CD39, CD47, PD1, PDL1, CD3, NKG2D, NKG2A, Nkp46, CD137, OX40, CD40, LILRB1, LILRB2, LILRB4, GPC3, TROP2, CD112, TIGIT, FAP, VEGFA, DLL4, ANG-2, wherein the first arm and the second arm specifically bind to different antigens.

In some embodiments, the Fc region of the first heavy chain constant region and/or the Fc region of the second heavy chain constant region further comprises modifications improving the stability of the antibody or the antigen-binding fragment.

In some embodiments, the antibody or an antigen-binding fragment thereof is humanized or chimeric antibody. In some embodiments, the antibody or an antigen-binding fragment thereof is a bispecific antibody or a multi-specific antibody.

In some embodiments, the antibody or an antigen-binding fragment thereof is linked to one or more conjugate moieties.

In some embodiments, the conjugate moiety comprises a second antibody fragment. In some embodiments, the antibody or antigen-binding fragment thereof comprises an Fab fragment, which is linked to the C-terminus of the Fc region(s) of the second antibody fragment.

In some embodiments, the conjugate moiety comprises an agent for detection or isolation, such as clearance-modifying agent, a luminescent label, a fluorescent label, an enzyme-substrate label, or a purification moiety. In some embodiments, the conjugate moiety comprises a therapeutic agent or a drug.

In one respect, the present disclosure provides an isolated polynucleotide encoding the antibody or an antigen-binding fragment thereof disclosed herein.

In one respect, the present disclosure provides a vector comprising the isolated polynucleotide disclosed herein.

In one respect, the present disclosure provides a host cell comprising the vector disclosed herein.

In one respect, the present disclosure provides a pharmaceutical composition, comprising: (i) the antibody or an antigen-binding fragment thereof, or the polynucleotide disclosed herein, and (ii) one or more pharmaceutically acceptable carriers.

In one respect, the present disclosure provides a method of expressing the antibody or an antigen-binding fragment thereof disclosed herein, comprising culturing the host cell disclosed herein under a condition suitable for expressing the vector contained therein.

In one respect, the present disclosure provides a method of treating, preventing or alleviating a disease in a subject, comprising administering to the subject a therapeutically effective amount of the antibody or an antigen-binding fragment thereof, or of the polynucleotide encoding the antibody or antigen-binding fragment thereof, or the vector, or the host cell, or the pharmaceutical composition disclosed herein.

In some embodiments, the subject is human. In some embodiments, the administration is via oral, nasal, intravenous, subcutaneous, sublingual, or intramuscular administration.

In one respect, the present disclosure provides use of the antibody or an antigen-binding fragment thereof, or of the polynucleotide encoding the antibody or antigen-binding fragment thereof, or the vector, or the host cell, or the pharmaceutical composition disclosed herein in the manufacture of a medicament for treating, preventing or alleviating a disease in a subject.

### BRIEF DESCRIPTION OF THE DRAWINGS

FIG. 1 shows the schematic diagram of screening assay 1.

FIG. 2 shows the schematic diagram of screening assay 2.

FIGS. 3A to 3D shows the structures of expression vectors used in screening assays 1 and 2. FIG. 3A shows the structure of expression vector for the light chain with or without mutations for shifted disulfide bonds. FIG. 3B shows the structure of expression vector for the heavy chain with or without mutations for shifted disulfide bonds. FIG. 3C shows the structure of expression vector for wildtype light chain with a VHH domain fused at the N-terminus. FIG. 3D shows the structure of expression vector for naked Fc with knob or hole residue substitutions.

FIGS. 4A and 4B show the results for relative abundance of the engineered asymmetric Fab-Fc and VHH-Fab-Fc variants characterized with SEC-HPLC. FIG. 4A shows the asymmetric Fab-Fc variants with different residue substitution pairs for CH1-CL interchain disulfide bond shift, as detected by screening assay 1. FIG. 4B shows the asymmetric VHH-Fab-Fc molecules with native CH1-CL interchain disulfide bond, as detected by screening assay 2.

FIG. 5 shows the schematic structure of the symmetric bivalent monospecific antibody with shifted interchain disulfide bond in both Fab arms.

FIGS. 6A and 6B show the results of yields (FIG. 6A) and monomer percentage evaluation (FIG. 6B) of the engineered symmetric antibody variants with different residue substitution pairs for CH1-CL interchain disulfide bond shift.

FIGS. 7A to 7D show the results for binding activity evaluation of all the symmetric bivalent anti-CLDN18.2 antibody variant binding against CLDN18.2-overexpressing MC38 cells by FACS assay.

FIG. 8 shows the results for thermostability analysis of the symmetric bivalent antibody variants with different residue substitution pairs for CH1-CL interchain disulfide bond shift using Thermo Shift Assay.

FIG. 9 shows the schematic diagram of Case 1 study.

FIG. 10 shows the results of assembly accuracy and thermostability of the antibodies tested in Case 1 study, as measured by LC-MS assay and differential scanning fluorimetry, respectively.

FIG. 11 shows the schematic diagram of Case 2 study.

FIG. 12 shows the results of assembly accuracy and thermostability of the antibodies tested in Case 2 study, as measured by LC-MS assay and differential scanning fluorimetry, respectively.

FIGS. 13A and 13B show SDS-PAGE results of the antibodies tested in Case 1 study (FIG. 13A) and Case 2 study (FIG. 13B).

FIG. 14 shows the SDS-PAGE and SEC results of the tested antibody ACF_361.

FIG. 15 shows the LC-MS results of the test antibody ACF_361.

FIG. 16A shows the binding affinity results of the antibodies to MC38/CD47/CLDN18.2 cells by FACS. FIG. 16B shows the binding affinity results of the antibodies to CHO/SIRPaVI cells by FACS. FIG. 16C shows the phagocytosis activity results of the antibodies.

FIG. 17 shows the SDS-PAGE and SEC results of the tested antibody ACF_389.

FIGS. 18 and 19 show Mono S chromatography purification results of the tested antibody ACF_389.

FIG. 20 shows the LC-MS result of the test antibody ACF_389.

FIG. 21A shows the binding affinity results of the antibodies to Raji/PDL1 cells by FACS. FIG. 21B shows the binding affinity results of the antibodies to CHO/SIRPαV1 cells by FACS.

FIGS. 22 and 23 show Mono S chromatography purification results of the tested antibody ESB07.451.

FIG. 24 shows the LC-MS result of the test antibody ESB07.451.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to a person skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety. The terminology used herein is for the purpose of describing particular embodiments only and is not intended to be limiting of the invention.

As used herein, the singular forms “a”, “an” and “the” are intended to include the plural forms as well, unless the context clearly indicates otherwise. By way of example, “an antibody” means one antibody or more than one antibody.

Furthermore, to the extent that the terms “including”, “includes”, “having”, “has”, “with”, or variants thereof are used in either the detailed description and/or the claims, such terms are intended to be inclusive in a manner similar to the term “comprising”. Throughout this disclosure, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

Reference throughout this disclosure to “one embodiment,” “an embodiment,” “a particular embodiment,” “some embodiment,” or “a certain embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

### I. Antibody Modification

In some aspects, the present disclosure provides modified antibodies with higher assembly accuracies and stability of the polypeptide chains of the antibodies.

In some aspects, the modified antibodies are designed for reducing the mispairing of heavy chains and light chains in bispecific or multi-specific antibodies having two or more different antigen-binding sites.

In some aspects, the modified antibodies have amino acid residue mutations wherein non-cysteine residues are mutated to cysteine residues to form new disulfide bonds between a heavy chain and a light chain of the antibody.

In some aspects, the modified antibodies have amino acid residue mutations wherein cysteine residues are mutated to non-cysteine residues to disrupt disulfide bond formation among the cysteine residues.

The terms “polypeptide”, “peptide”, and “protein” are used interchangeably herein to designate a linear series of amino acid residues connected one to the other by peptide bonds, which includes proteins, polypeptides, oligopeptides, peptides, and fragments thereof. The protein may be made up of naturally occurring amino acids and/or synthetic (e.g., modified or non-naturally occurring) amino acids. Thus “amino acid”, or “peptide residue”, as used herein means both naturally occurring and synthetic amino acids. The terms “polypeptide”, “peptide”, and “protein” includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like. Furthermore, it should be noted that a dash at the beginning or end of an amino acid residue sequence indicates either a peptide bond to a further sequence of one or more amino acid residues or a covalent bond to a carboxyl or hydroxyl end group. However, the absence of a dash should not be taken to mean that such peptide bonds or covalent bond to a carboxyl or hydroxyl end group is not present, as it is conventional in representation of amino acid sequences to omit such.

The term “antibody” as used herein includes any immunoglobulin, monoclonal antibody, polyclonal antibody, multivalent antibody, bivalent antibody, monovalent antibody, multi-specific antibody, bispecific antibody or mono-specific antibody that binds to one or more specific antigens. A native intact antibody comprises two heavy (H) chains and two light (L) chains. Mammalian heavy chains are classified as alpha, delta, epsilon, gamma, and mu, each heavy chain consists of a variable region (VH) and a first, second, third, and optionally fourth constant region (CH1, CH2, CH3, CH4 respectively); mammalian light chains are classified as λ or κ, while each light chain consists of a variable region (VL) and a constant region. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, HCDR3). CDR boundaries for the antibodies and antigen-binding fragments disclosed herein may be defined or identified by the conventions of Kabat, IMGT, Chothia, or Al-Lazikani (Al-Lazikani, B., Chothia, C., Lesk, A. M., J. Mol. Biol., 273 (4), 927 (1997); Chothia, C. et al., J Mol Biol. December 5; 186 (3): 651-63 (1985); Chothia, C. and Lesk, A. M., J. Mol. Biol., 196,901 (1987); Chothia, C. et al., Nature. December 21-28; 342 (6252): 877-83 (1989); Kabat E. A. et al., Sequences of Proteins of immunological Interest, 5th Ed. Public Health Service, National Institutes of Health, Bethesda, Md. (1991); Marie-Paule Lefranc et al., Developmental and Comparative Immunology, 27:55-77 (2003); Marie-Paule Lefranc et al., Immunome Research, 1 (3), (2005); Marie-Paule Lefranc, Molecular Biology of B cells (second edition), chapter 26, 481-514, (2015)). The three CDRs are interposed between flanking stretches known as framework regions (FRs) (light chain FRs including LFR1, LFR2, LFR3, and LFR4, heavy chain FRs including HFR1, HFR2, HFR3, and HFR4), which are more highly conserved than the CDRs and form a scaffold to support the highly variable loops. The constant regions of the heavy and light chains are not involved in antigen-binding, but exhibit various effector functions. Antibodies are assigned to classes based on the amino acid sequences of the constant regions of their heavy chains. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgAQ1 (alpha1 heavy chain), or IgA2 (alpha2 heavy chain).

The term “variable domain” with respect to an antibody as used herein refers to an antibody variable region or a fragment thereof comprising one or more CDRs. Although a variable domain may comprise an intact variable region (such as HCVR or LCVR), it is also possible to comprise less than an intact variable region yet still retain the capability of binding to an antigen or forming an antigen-binding site.

The term “antigen-binding fragment” as used herein refers to an antibody fragment formed from a portion of an antibody comprising one or more CDRs, or any other antibody fragment that binds to an antigen but does not comprise an intact native antibody structure. Examples of antigen-binding fragment include, without limitation, a variable domain, a variable region, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a multispecific antibody, a camelized single domain antibody, a nanobody, a domain antibody, and a bivalent domain antibody. An antigen-binding fragment is capable of binding to the same antigen to which the parent antibody binds. In certain embodiments, an antigen-binding fragment may comprise one or more CDRs from a particular human antibody grafted to a framework region from one or more different human antibodies. For more and detailed formats of antigen-binding fragment (also called as antigen-binding moiety) are described in Spiess et al, 2015 Molecular Immunology, 67(2), pp. 95-106 (2015), and Brinkman et al., mAbs, 9(2), pp. 182-212 (2017), which are incorporated herein by entirety reference.

“Fab” with regard to an antibody refers to that portion of the antibody consisting of a single light chain (both variable and constant regions) bound to the variable region and first constant region of a single heavy chain by a disulfide bond.

“Fab′” refers to an Fab fragment that includes a portion of the hinge region.

“F(ab′)₂” refers to a dimer of Fab′.

“Fc” with regard to an antibody (e.g., of IgG, IgA, or IgD isotype) refers to that portion of the antibody consisting of the second and third constant domains of a first heavy chain bound to the second and third constant domains of a second heavy chain via disulfide bonding. Fc with regard to antibody of IgM and IgE isotype further comprises a fourth constant domain. The Fc portion of the antibody is responsible for various effector functions such as antibody-dependent cell-mediated cytotoxicity (ADCC), and complement dependent cytotoxicity (CDC), but does not function in antigen binding.

“Hinge region” in terms of an antibody includes the portion of a heavy chain molecule that joins the CH1 domain to the CH2 domain. This hinge region comprises approximately 25 amino acid residues and is flexible, thus allowing the two N-terminus antigen binding regions to move independently.

“CH2 domain” as used herein refers to includes the portion of a heavy chain molecule that extends, e.g., from about amino acid 244 to amino acid 360 of an IgG antibody using conventional numbering schemes (amino acids 244 to 360, Kabat numbering system; and amino acids 231-340, EU numbering system; see Kabat, E., et al., U.S. Department of Health and Human Services, (1983)).

The CH3 domain extends from the CH2 domain to the C-terminus of the IgG molecule and comprises approximately 108 amino acids. Certain immunoglobulin classes, e.g., IgM, further include a CH4 region.

“Fv” with regard to an antibody refers to the smallest fragment of the antibody to bear the complete antigen binding site. An Fv fragment consists of the variable region of a single light chain bound to the variable region of a single heavy chain. A number of Fv designs have been provided, including dsFvs, in which the association between the two domains is enhanced by an introduced disulphide bond; and scFvs can be formed using a peptide linker to bind the two domains together as a single polypeptide. Fv constructs containing a variable domain of a heavy or light immunoglobulin chain associated to the variable and constant domain of the corresponding immunoglobulin heavy or light chain have also been produced. Fvs have also been multimerised to form diabodies and triabodies (Maynard et al., Annu Rev Biomed Eng 2 339-376 (2000)).

“Camelized single domain antibody,” “heavy chain antibody,” or “HCAb” refers to an antibody that contains two V_{H} domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363(6428): 446-8 (1993); Nguyen V K. et al. Immunogenetics. April; 54(1):39-47 (2002); Nguyen V K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 Jun. 15 (2007)).

A “nanobody” refers to an antibody fragment that consists of a VHH domain from a heavy chain antibody and two constant domains, CH2 and CH3.

A “diabody” or “dAb” includes small antibody fragments with two antigen-binding sites, wherein the fragments comprise a V_{H} domain connected to a V_{L} domain in the same polypeptide chain (V_{H}-V_{L} or V_{L}-V_{H}) (see, e.g. Holliger P. et al., Proc Natl Acad Sci USA. July 15; 90 (14): 6444-8 (1993); EP404097; WO93/11161). By using a linker that is too short to allow pairing between the two domains on the same chain, the domains are forced to pair with the complementarity domains of another chain, thereby creating two antigen-binding sites. The antigen-binding sites may target the same or different antigens (or epitopes). In certain embodiments, a “bispecific ds diabody” is a diabody targeting two different antigens (or epitopes).

The term “valent” as used herein refers to the presence of a specified number of antigen binding sites in a given molecule. The term “monovalent” refers to an antibody or an antigen-binding fragment having only one single antigen-binding site; and the term “multivalent” refers to an antibody or an antigen-binding fragment having multiple antigen-binding sites. As such, the terms “bivalent”, “trivalent”, “tetravalent”, and “hexavalent” denote the presence of two binding sites, three binding sites, four binding sites, and six binding sites, respectively, in an antigen-binding molecule. In some embodiments, the antibody or antigen-binding fragment thereof is bivalent.

As used herein, a “bispecific” antibody refers to an artificial antibody which has fragments derived from two different monoclonal antibodies and is capable of binding to two different epitopes. The two epitopes may present on the same antigen, or they may present on two different antigens.

As used herein, a “multi-specific” antibody refers to an artificial antibody which has fragments derived from more than two different monoclonal antibodies and is capable of binding to more than two different epitopes. The more than two epitopes may present on the same antigen, or they may present on the more than two different antigens.

The term “chimeric” as used herein, means an antibody or antigen-binding fragment, having a portion of heavy and/or light chain derived from one species, and the rest of the heavy and/or light chain derived from a different species. In an illustrative example, a chimeric antibody may comprise a constant region derived from human and a variable region from a non-human animal, such as from mouse. In some embodiments, the non-human animal is a mammal, for example, a mouse, a rat, a rabbit, a goat, a sheep, a guinea pig, or a hamster.

The term “humanized” as used herein means that the antibody or antigen-binding fragment comprises CDRs derived from non-human animals, FR regions derived from human, and when applicable, the constant regions derived from human.

The term “affinity” as used herein refers to the strength of non-covalent interaction between an immunoglobulin molecule (i.e., antibody) or fragment thereof and an antigen.

The term “specific binding” or “specifically binds” as used herein refers to a non-random binding reaction between two molecules, such as for example between an antibody and an antigen. Specific binding can be characterized in binding affinity, for example, represented by K_{D} value, i.e., the ratio of dissociation rate to association rate (k_{off}/kₒₙ) when the binding between the antigen and antigen-binding molecule reaches equilibrium. K_{D} may be determined by using any conventional method known in the art, including but are not limited to, surface plasmon resonance method, microscale thermophoresis method, HPLC-MS method and flow cytometry (such as FACS) method. A K_{D} value of ≤10⁻⁶ M (e.g. ≤5×10⁻⁷ M, ≤2×10⁻⁷ M, ≤10⁻⁷ M, ≤5×10⁻⁸ M, ≤2×10⁻⁸ M, ≤10⁻⁸ M, ≤5×10⁻⁹ M, ≤4×10⁻⁹M, ≤3×10⁻⁹M, ≤2×10⁻⁹ M, or ≤10⁻⁹ M) can indicate specific binding between an antibody or antigen binding fragments thereof and the corresponding antigen.

The term “epitope” as used herein refers to the specific group of atoms or amino acids on an antigen to which an antibody binds. Two antibodies may bind the same or a closely related epitope within an antigen if they exhibit competitive binding for the antigen. An epitope can be linear or conformational (i.e., including amino acid residues spaced apart).

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). In other words, percent (%) sequence identity of an amino acid sequence (or nucleic acid sequence) can be calculated by dividing the number of amino acid residues (or bases) that are identical relative to the reference sequence to which it is being compared by the total number of the amino acid residues (or bases) in the candidate sequence or in the reference sequence, whichever is shorter. Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol., 215:403-410 (1990); Stephen F. et al., Nucleic Acids Res., 25:3389-3402 (1997)), ClustalW2 (available on the website of European Bioinformatics Institute, see also, Higgins D. G. et al., Methods in Enzymology, 266:383-402 (1996); Larkin M. A. et al., Bioinformatics (Oxford, England), 23 (21): 2947-8 (2007)), and ALIGN or Megalign (DNASTAR) software. A person skilled in the art may use the default parameters provided by the tool, or may customize the parameters as appropriate for the alignment, such as for example, by selecting a suitable algorithm.

The term “mutation”, “mutated” with regard to amino acid residues as used herein refers to substitution, insertion, or deletion of one or more amino acid residues.

The term “substitution” or “substituted” with regard to amino acid sequence as used herein refers to replacement of a native amino acid residue using a different amino acid residue.

The term “conservative substitution” with regard to amino acid sequence refers to replacing an amino acid residue with a different amino acid residue having a side chain with similar physiochemical properties. For example, conservative substitutions can be made among amino acid residues with hydrophobic side chains (e.g., Met, Ala, Val, Leu, and Ile), among amino acid residues with neutral hydrophilic side chains (e.g., Cys, Ser, Thr, Asn and Gln), among amino acid residues with acidic side chains (e.g., Asp, Glu), among amino acid residues with basic side chains (e.g., His, Lys, and Arg), or among amino acid residues with aromatic side chains (e.g., Trp, Tyr, and Phe). As known in the art, conservative substitution usually does not cause significant change in the protein conformational structure, and therefore could retain the biological activity of a protein.

The term “subject” includes human and non-human animals. Non-human animals include all vertebrates, e.g., mammals and non-mammals, such as non-human primates, mice, rats, cats, rabbits, sheep, dogs, cows, chickens, amphibians, and reptiles. Except when noted, the terms “patient” or “subject” are used herein interchangeably.

The antibodies or antigen binding fragments thereof may comprise one or more modifications that introduce or remove a glycosylation site. A glycosylation site is an amino acid residue with a side chain to which a carbohydrate moiety (e.g., an oligosaccharide structure) can be attached. Glycosylation of antibodies is typically either N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine.

### Formation and Disruption of Disulfide Bonds

In some embodiments, the antibodies provided herein includes a first arm and a second arm, wherein the first arm is formed by a first heavy chain and a first light chain with at least one disulfide bond therebetween, and the second arm formed by a second heavy chain and a second light chain with at least one disulfide bond therebetween.

In some embodiments, the connection between the first heavy chain and the first light chain forming the first arm includes artificially introduced disulfide bond(s), while the connection between the second heavy chain and the second light chain forming the second arm includes naturally occurring disulfide bonds. In some embodiments, the connection between the first heavy chain and the first light chain forming the first arm includes artificially introduced disulfide bond(s) at a first set of positions, while the connection of the second heavy chain and the second light chain forming the second arm includes artificially introduced disulfide bond(s) at a second set of positions which is different from the first set of positions.

In some aspects, a modified antibody has amino acid residue mutations wherein a pair of non-cysteine residues (which can be any amino acid other than a cysteine) located on a heavy chain and a light chain of the antibody, respectively, are mutated to cysteine residues to form new disulfide bonds between them. In certain embodiments, the non-cysteine residue on the heavy chain is located at the amino acid position of 126, 128, 129, 136, 141, 168, 170, 173, 175 or 187 according to EU numbering, and the non-cysteine residue on the light chain is located at the amino acid position of 114, 116, 118, 124, 135, 137, 138, 160, 162 or 164 according to EU numbering. In certain embodiments, the non-cysteine residue on the heavy chain is the amino acid residue of F126, L128, A129, S136, A141, H168, F170, V173, Q175 or T187 according to EU numbering. In certain embodiments, the non-cysteine residue on the light chain is the amino acid residue of S114, F116, F118, Q124, L135, N137, N138, Q160, S162 or T164 according to EU numbering.

In certain embodiments, the modified antibodies include one or more pairs of mutation from non-cysteine residues to cysteine residues at the following positions:

TABLE 1
Amino acid residues to be mutated from non-
cysteine residues to cysteine residues.

| Position in light chain | Position in heavy chain |
|---|---|
| (EU numbering) | (EU numbering) |
| 114 | 136 |
| 116 | 136 |
| 118 | 128 |
| 118 | 129 |
| 118 | 141 |
| 124 | 126 |
| 135 | 170 |
| 137 | 170 |
| 138 | 168 |
| 160 | 173 |
| 160 | 175 |
| 162 | 170 |
| 162 | 187 |
| 164 | 170 |

In certain embodiments, the modified antibodies include one or more pairs of the following pairs of mutation from non-cysteine residues to cysteine residues:

TABLE 2
Amino acid residues to be mutated from non-
cysteine residues to cysteine residues.

| Position in light chain | Position in heavy chain |
|---|---|
| (EU numbering) | (EU numbering) |
| S114C | S136C |
| F116C | S136C |
| F118C | L128C |
| F118C | A129C |
| F118C | A141C |
| Q124C | F126C |
| L135C | F170C |
| N137C | F170C |
| N138C | H168C |
| Q160C | V173C |
| Q160C | Q175C |
| S162C | F170C |
| S162C | T187C |
| T164C | F170C |

In some aspects, the modified antibodies have amino acid residue mutations wherein cysteine residues are mutated to non-cysteine residues to disrupt disulfide bond formation among the cysteine residues. In certain embodiments, the pair of cysteine residues mutated to non-cysteine residues are amino acid reside C214 in the light chain and amino acid residue C220 in the heavy chain of the antibody.

In certain embodiments, the modified antibodies have one or more pair of amino acid residue mutations at the positions selected from Table 1 or Table 2, and the pair of cysteine residues mutated to non-cysteine residues are amino acid reside C214 in the light chain and amino acid residue C220 in the heavy chain of the antibody.

In some embodiments, the antibody or antigen-binding fragment thereof of the present disclosure includes a first arm formed by a first heavy chain and a first light chain with two new disulfide bonds formed therebetween, wherein the two disulfide bonds are formed by the pairs of mutations from non-cysteine to cysteine selected from the mutation pairs listed in Table 1 or Table 2.

In certain embodiments, an antibody or antigen-binding fragment thereof of the present disclosure includes a first arm formed by a first heavy chain and a first light chain with a disulfide bond formed therebetween, and a second arm formed by a second heavy chain and a second light chain with a disulfide bond formed therebetween, wherein the first light chain and the first heavy chain includes at least one pair of mutation from non-cysteine residue to cysteine residue selected from the group consisting of:
(i) S114C on light chain and S136C on heavy chain;
(ii) F116C on light chain and S136C on heavy chain;
(iii) F118C on light chain and L128C on heavy chain;
(iv) F118C on light chain and A129C on heavy chain;
(v) F118C on light chain and A141C on heavy chain;
(vi) Q124C on light chain and F126C on heavy chain;
(vii) L135C on light chain and F170C on heavy chain;
(viii) N137C on light chain and F170C on heavy chain;
(ix) N138C on light chain and H168C on heavy chain;
(x) Q160C on light chain and V173C on heavy chain;
(xi) Q160C on light chain and Q175C on heavy chain;
(xii) S162C on light chain and F170C on heavy chain;
(xiii) S162C on the light chain and T187C on heavy chain; and
(xiv) T164C on light chain and F170C on heavy chain;
wherein the at least one pair of mutation from non-cysteine residue to cysteine residue forms at least one disulfide bond between the first heavy chain and the first light chain, and the native cysteine on the first light chain and the native cysteine on the first heavy chain is mutated to a non-cysteine amino acid. In certain embodiments, the non-cysteine residue that the native cysteine mutated into is independently selected from S, A or G.

All the numberings of amino acid residues and positions on the heavy chain and light chain of antibodies described in the present disclosure are according to EU numbering system unless otherwise specified. As used herein, the term “EU numbering system” refers to the EU numbering convention for the constant regions of an antibody, as described in Edelman, G. M. et al., Proc. Natl. Acad. USA, 63, 78-85 (1969) and Kabat et al, Sequences of Proteins of Immunological Interest, U.S. Dept. Health and Human Services, 5th edition, 1991, each of which is herein incorporated by reference in its entirety.

In some embodiments, the naturally occurring disulfide bond(s) formed between the native cysteine on the second light chain CL region and the native cysteine on the second heavy chain CH1 region is disrupted by non-cysteine substitutions on both cysteines. In some embodiments, the native cysteines are independently substituted by any one of non-cysteine residues, as long as the artificially introduced new pair of residues does not have any interaction therebetween. In some embodiments, the native cysteines are independently substituted by serine(S), alanine (A) or glycine (G). In some embodiments, the native cysteines are both substituted by serine(S).

In some embodiments, the artificially introduced disulfide bond(s) is formed by the mutant combination selected from the group consisting of the mutations listed in Table 3.

TABLE 3
Mutation sites on CH1 and CL to form the artificially
introduced disulfide bonds between CH1 and CL.

| Mutant site in CL | Mutant site in CH1 |
|---|---|
| S114C/C214S | S136C/C220S |
| F116C/C214S | S136C/C220S |
| F118C/C214S | L128C/C220S |
| F118C/C214S | A129C/C220S |
| F118C/C214S | A141C/C220S |
| Q124C/C214S | F126C/C220S |
| Q160C/C214S | Q175C/C220S |
| Q160C/C214S | V173C/C220S |
| L135C/C214S | F170C/C220S |
| N137C/C214S | H170C/C220S |
| N138C/C214S | H168C/C220S |
| S162C/C214S | F170C/C220S |
| S162C/C214S | T187C/C220S |
| T164C/C214S | F170C/C220S |
| S114C/Q160C/C214S | S136C/V173C/C220S |
| F118C/Q124C/C214S | L128C/F126C/C220S |
| F118C/Q124C/C214S | A129C/F126C/C220S |
| F118C/Q124C/C214S | A141C/F126C/C220S |
| F116C/Q124C/C214S | S136C/F126C/C220S |
| Q124C/T164C/C214S | F126C/F170C/C220S |
| Q124C/N138C/C214S | F126C/H168C/C220S |
| Q160C/L135C/C214S | Q175C/F170C/C220S |
| S114C/S162C/C214S | S136C/F170C/C220S |
| F118C/S162C/C214S | L128C/F170C/C220S |
| F118C/S162C/C214S | A141C/F170C/C220S |
| Q124C/S162C/C214S | F126C/F170C/C220S |
| N137C/S162C/C214S | F170C/T187C/C220S |

In some embodiments, the connection between the first heavy chain and the first light chain forming the first arm comprises two pairs of artificially introduced disulfide bond formed by the mutant combination selected from the group consisting of:
(i) S114C/Q160C on light chain and S136C/V173C on heavy chain;
(ii) F118C/Q124C on light chain and L128C/F126C on heavy chain;
(iii) F118C/Q124C on light chain and A129C/F126C on heavy chain;
(iv) F118C/Q124C on light chain and A141C/F126C on heavy chain;
(v) F116C/Q124C on light chain and S136C/F126C on heavy chain;
(vi) Q124C/T164C on light chain and F126C/F170C on heavy chain;
(vii) Q124C/N138C on light chain and F126C/H168C on heavy chain;
(viii) Q160C/L135C on light chain and Q175C/F170C on heavy chain;
(ix) S114C/S162C on light chain and S136C/F170C on heavy chain;
(x) F118C/S162C on light chain and L128C/F170C on heavy chain;
(xi) F118C/S162C on light chain and A141C/F170C on heavy chain;
(xii) Q124C/S162C on light chain and F126C/F170C on heavy chain; and
(xiii) N137C/S162C on light chain and F170C/T187C on heavy chain.

In some embodiments, the connection between the first heavy chain and the first light chain forming the first arm comprises two pairs of artificially introduced disulfide bond formed by the mutant combination selected from the group consisting of:
(i) F118C/Q124C on light chain and L128C/F126C on heavy chain;
(ii) Q124C/T164C on light chain and F126C/F170C on heavy chain;
(iii) F118C/S162C on light chain and L128C/F170C on heavy chain;
(iv) F118C/S162C on light chain and A141C/F170C on heavy chain; and
(v) Q124C/S162C on light chain and F126C/F170C on heavy chain.

In some embodiments, the antibodies or antigen-binding fragments thereof disclosed herein comprises a heavy chain CH1 region that is at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to a human germline heavy chain CH1 region. In some embodiments, the human germline heavy chain CH1 region has the amino acid sequence set forth in SEQ ID NO:5 provided herein.

In some embodiments, the antibodies or antigen-binding fragments thereof disclosed herein comprises a light chain CL region that is at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to a human germline light chain CL region. In some embodiments, the human germline light chain CL region has the amino acid sequence set forth in SEQ ID NO:4 provided herein.

### Formation of Bispecific Antibodies

By relocating the disulfide bonds formed by pairs of cysteines from a CH1 region and a CL region, the present disclosure provides modified antibodies whose heavy chains and light chains are paired with high assembly accuracies and stability.

As used herein, “bispecific antibodies” are antibodies that have binding specificities for at least two independent antigens or different epitopes within the same antigen.

In certain embodiments, bispecific antibodies include a first binding site for one epitope and a second binding site for another epitope. In certain embodiments, the two antigen targets are selected from the group consisting of: SIRPα, CLDN18.2, Siglec15, HER2, EGFR, CD19, CD20, CD39, CD47, PD1, PDL1, CD3, NKG2D, NKG2A, Nkp46, CD137, OX40, CD40, LILRB1, LILRB2, LILRB4, GPC3, TROP2, CD112, TIGIT, FAP, VEGFA, DLL4, ANG-2. In certain embodiments, one of the antigen binding arms of the bispecific antibody may target to SIRPα, and the other antigen binding arms of the bispecific antibody may target to Claudin 18.2.

In certain embodiments, the binding affinities for two independent antigens (or epitopes on the same antigen) of the bispecific antibody are about the same. In certain embodiments, the binding affinities for two independent antigens (or epitopes on the same antigen) of the bispecific antibody are different. In some embodiments, the affinities for the two independent antigens (or epitopes on the same antigen) of the bispecific antibody may differ by 1 fold, 2 folds, 3 folds, 4 folds or more.

### Mutations on Fc Regions

The antibodies or antigen-binding fragments thereof disclosed herein also encompass Fc variants, which may comprise one or more amino acid residue modifications or substitutions at the Fc region and/or hinge region.

In some embodiments, heterodimeric pairing is achieved by engineering the Fc regions of two heavy chains so that it forms a heterodimer exclusively. In some embodiments, the CH3 regions of the Fc regions are introduced with mutations.

In certain embodiments, the antibodies or antigen-binding fragments thereof comprise one or more amino acid substitution(s) in the interface of the Fc region to facilitate and/or promote heterodimerization. These modifications comprise introduction of a protuberance into a first Fc polypeptide and a cavity into a second Fc polypeptide, wherein the protuberance can be positioned in the cavity so as to promote interaction of the first and second Fc polypeptides to form a heterodimer or a complex. Methods of generating antibodies with these modifications are known in the art, e.g. as described in U.S. Pat. No. 5,731,168.

In some embodiments, a “Knob” is generated by replacing one or more small amino acid side chains from the interface of the first antibody molecule with larger side chains (e.g., tyrosine or tryptophan). Compensatory “Holes” of identical or similar size to the large side chain(s) are created on the interface of the second antibody molecule by replacing large amino acid side chains with smaller ones (e.g., alanine or threonine). CH3 modifications to enhance heterodimerization include, for example, Y407V/T366S/L368A on one heavy chain and T366W on the other heavy chain; S354C/T366W on one heavy chain and Y349C/Y407V/T366S/L368A on the other heavy chain. Such modifications resulting in a protrusion on one chain and a cavity on the other are provided in Table 4.

TABLE 4
Mutations forming Knob and Hole structures
for Fc heterodimerization.

| Modification(s) in one | Modification(s) in the other one |
|---|---|
| CH3 region | CH3 region |
| T366S/L368A/Y407V | T366W |
| S354C/T366W | Y349C/T366S/L368A/Y407V |
| T366Y | Y407T |
| T366W | Y407A |
| T394W | F405A |
| T366Y/F405A | T394W/Y407T |
| T366W/F405W | T394S/Y407A |
| F405W | T394S |
| D399C | K392C |
| T366W/D399C | T3 66S/L368A/K392C/Y407V |
| T366W/K392C | T366S/L368A/D399C/Y407V |
| S354C/T366W | Y349C/T366S/L368A/Y407V |
| Y349C/T366W | S354C/T366S/L368A/Y407V |
| E356C/T366W | Y349C/T366S/L368A/Y407V |
| Y349C/T366W | E356C/T366S/L368A/Y407V |
| E357C/T366W | Y349C/T366S/L368A/Y407V |
| Y349C/T366W | E357C/T366S/L368A/Y407V |

In some embodiments, the antibody or antigen-binding fragments thereof disclosed herein comprises a first CH3 region and a second CH3 region, wherein the first CH3 region or the second CH3 region comprises an amino acid sequence differing from wild-type IgG amino acid sequence such that one or more positive-charged amino acids (e.g., lysine, histidine and arginine) in the wild-type human IgG amino acid sequence are replaced with one or more negative-charged amino acids (e.g., aspartic acid and glutamic acid) at the corresponding position(s) in the CH3 region. Alternatively, the first CH3 region or the second CH3 region comprises van amino acid sequence differing from wild-type IgG amino acid sequence such that one or more negative-charged amino acids in the wild-type human IgG amino acid sequence are replaced with one or more positive-charged amino acids at the corresponding position(s) in the CH3 region. In some embodiments, the modifications in the two CH3 regions are selected from the group listed in Table 5.

TABLE 5
Mutations altering the charge polarity for Fc heterodimerization.

| Modification(s) in one | Modification(s) in the other one |
|---|---|
| CH3 region | CH3 region |
| K370E/D399K/K439D | D356K/E357K/K409D |
| K409D | D399K |
| K409E | D399K |
| K409E | D399R |
| K409D | D399R |
| D339K | E356K |
| E356K/D399K | K392D/K409D |
| E356K/D399K | K409D/K439D |
| E357K/D399K | K370D/K409D |
| E356K/E357K/D399K | K370D/K392D/K409D |
| E357K/D399K | K392D/K409D |
| K392D/K409D | D399K |
| K360D/K409D | D399K |

In some embodiments, the heavy chain constant regions of the antibody or antigen-binding fragments thereof can be derived from human IgG1, IgG2, IgG3 or IgG4. In some embodiments, the light chain constant regions of the antibody or antigen-binding fragments thereof can be derived from human kappa chain or human lambda chain.

### II. Conjugates

In some embodiments, the antibodies or antigen-binding fragments thereof further comprise one or more conjugate moieties. In certain embodiments, the antibodies or antigen-binding fragments thereof provided herein is used as a base for a conjugate.

The conjugate moiety can be linked to the antibodies or antigen-binding fragments thereof. A conjugate moiety is a moiety that can be attached to the antibody or antigen-binding fragment thereof. It is contemplated that a variety of conjugate moieties may be linked to the antibodies or antigen-binding fragments thereof provided herein (see, for example, “Conjugate Vaccines”, Contributions to Microbiology and Immunology, J. M. Cruse and R. E. Lewis, Jr. (eds.), Carcer Press, New York, (1989)). These conjugate moieties may be linked to the antibodies or antigen-binding fragments thereof by covalent binding, affinity binding, intercalation, coordinate binding, complexation, association, blending, or addition, among other methods. In some embodiments, the antibodies or antigen-binding fragments thereof can be linked to one or more conjugates via a linker.

### III. Method of Preparation

The present disclosure provides polynucleotides that encode the antibodies or antigen-binding fragments thereof provided herein.

The term “polynucleotide” or “nucleic acid” as used herein refers to deoxyribonucleic acids (DNA) or ribonucleic acids (RNA) and polymers thereof in either single- or double-stranded form. Unless otherwise indicated, a particular polynucleotide sequence also implicitly encompasses conservatively modified variants thereof (e.g., degenerate codon substitutions), alleles, orthologs, SNPs, and complementarity sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The nucleic acids or polynucleotides encoding the antibodies or antigen-binding fragments thereof provided herein can be constructed using recombinant techniques. To this end, DNA encoding an antigen-binding fragment of a parent antibody (such as CDR or variable region) can be isolated and sequenced using conventional procedures (e.g., by using oligonucleotide probes that are capable of binding specifically to genes encoding the heavy and light chains of the antibody). As an example, the polynucleotide sequence encoding the variable domain (VH) and the polynucleotide sequence encoding CH1, CH2 and CH3 are obtained and operably linked to allow transcription and expression in a host cell to produce the heavy chain polypeptide. Similarly, polynucleotide sequence encoding VL are operably linked to polynucleotide sequence encoding CL, so as to allow expression of the light chain in the host cell.

Mutations can be introduced by various methods known in the art. In some embodiments, primers with designed mutations (e.g., including point mutations, deletion of a segment of polynucleotides, or insertion of a segment of polynucleotides) can be used for introducing certain mutations, using PCR technology.

In some embodiments, the polynucleotides, with or without mutations, may be obtained by synthetic methods. Methods of chemical DNA synthesis are well known in the art. Typically, the methods include steps as following. The 5′-end of the first nucleotide is protected by dimethoxytrityl (DMT) while a linker to silica attaches the OH end. The reactive groups of all nucleotides are chemically protected. Afterwards DMT is removed by washing and the next nucleotide is activated and attached to the 3′—OH group. Using iodine, the 5′ to 3′ linkage is oxidized to generate a phosphotriester bond (one of the O of the phosphate group is methylated). The reaction is continued until the desired chain length is reached. About 70-80 residue polymers can be made this way. The process has also automated versions and routine synthetic services are commercially available. DNA encoding the antibodies or antigen-binding fragments thereof disclosed herein is readily isolated and sequenced using conventional procedures (e.g., by using oligonucleotide probes that are capable of binding specifically to genes encoding the heavy and light chains of the antibody).

The encoding polynucleotide sequences can be further operably linked to one or more regulatory sequences, optionally in an expression vector, such that the expression or production of the heavy chain and the light chain is feasible and under proper control.

The present disclosure provides vectors comprising the encoding polynucleotide provided herein. The term “vector” as used herein refers to a vehicle into which a polynucleotide encoding a protein may be operably inserted so as to bring about the expression of that protein. Typically, the construct also includes appropriate regulatory sequences. For example, the polynucleotide molecule can include regulatory sequences located in the 5′-flanking region of the nucleotide sequence encoding the guide RNA and/or the nucleotide sequence encoding a site-directed modifying polypeptide, operably linked to the coding sequences in a manner capable of expressing the desired transcript/gene in a host cell. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating.

Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the antibody or antigen-binding fragment thereof can be introduced to a host cell for cloning or gene expression. The term “host cell” as used herein refers to a cell into which an exogenous polynucleotide and/or a vector has been introduced.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g. E. coli, Enterobacter, Erwinia, Klebsiella, Proteus, Salmonella, e.g. Salmonella typhimurium, Serratia, e.g. Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g. K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g. Neurospora, Penicillium, Tolypocladium, and Aspergillus hosts such as A. nidulans and A. niger.

Suitable host cells for the expression of glycosylated antibodies or antigen-fragment thereof provided herein are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruit fly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present disclosure, particularly for transfection of Spodoptera frugiperda cells. Plant cell cultures of cotton, corn, potato, soybean, petunia, tomato, and tobacco can also be utilized as hosts.

In certain embodiments, host cells are vertebrate cells. Propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). In some embodiments, the host cell is a mammalian cultured cell line, such as CHO, BHK, NS0, 293 and their derivatives.

Host cells are transformed with the above-described expression or cloning vectors for antibody production and cultured in conventional nutrient media modified as appropriate for inducing promoters, selecting transformants, or amplifying the genes encoding the desired sequences.

The present disclosure also provides a method of expressing the antibodies or antigen-binding fragments thereof provided herein, comprising culturing the host cell provided herein under the condition at which the vector of the present disclosure is expressed. The host cells used to produce the antibodies or antigen-binding fragments thereof provided herein may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to a person skilled in the art. The culture conditions, such as temperature, pH, and the like, are those previously used with the host cell selected for expression, and will be apparent to a person skilled in the art.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

In certain embodiments, the present disclosure provides a method of producing the antibody or antigen-binding fragments thereof provided herein, comprising a) introducing to a host cell: a first polynucleotide encoding a first heavy chain, a second polynucleotide encoding a first light chain, a third polynucleotide encoding a second heavy chain, and a fourth polynucleotide encoding a second light chain, wherein the first light chain and the first heavy chain comprises a non-cysteine substitution pair of C214X on the first light chain and C220X on the first heavy chain and at least one cysteine substitution pair; b) allowing the host cell to express the polypeptide complex. In certain embodiments, the at least one cysteine substitution pair selected from the group consisting of: (i) S114C on light chain and S136C on heavy chain; (ii) F116C on light chain and S136C on heavy chain; (iii) F118C on light chain and L128C on heavy chain; (iv) F118C on light chain and A129C on heavy chain; (v) F118C on light chain and A141C on heavy chain; (vi) Q124C on light chain and F126C on heavy chain; (vii) L135C on light chain and F170C on heavy chain; (viii) N137C on light chain and F170C on heavy chain; (ix) N138C on light chain and H168C on heavy chain; (x) T164C on light chain and F170C on heavy chain; (xi) Q160C on light chain and V173C on heavy chain; (xii) Q160C on light chain and Q175C on heavy chain; and (xiii) S162C on light chain and F170C on heavy chain.

In certain embodiments, the method further comprises isolating the antibody or antigen-binding fragments thereof. The term “isolate” is intended to mean a compound of interest has been separated or purified from components that accompany it in nature or during manufacture and provided in an enriched form.

The antibodies or antigen-binding fragments thereof prepared from the cells can be isolated using, for example, hydroxylapatite chromatography, gel electrophoresis, dialysis, DEAE-cellulose ion exchange chromatography, ammonium sulfate precipitation, salting out, and affinity chromatography.

In certain embodiments, Protein A immobilized on a solid phase is used for immunoaffinity purification of the antibody and antigen-binding fragment thereof. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (Lindmark et al., J. Immunol. Meth. 62:1-13 (1983)). Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody or antigen-binding fragments thereof of interest and contaminants may be subjected to hydrophobic interaction chromatography or ion exchange chromatography using a gradient elution of salt concentrations.

In certain embodiments, the antibodies or antigen-binding fragments thereof provided herein can be readily purified with high yields using conventional methods. One of the advantages of the antibodies or antigen-binding fragments thereof is the significantly reduced mispairing between heavy chain and light chain variable domain sequences. This reduces production of unwanted byproducts and make it possible to obtain high purity product in high yields using relatively simple purification processes.

### IV. Pharmaceutical Compositions and Administration

The present disclosure further provides pharmaceutical compositions comprising the antibodies or antigen-binding fragments thereof and one or more pharmaceutically acceptable carriers.

The present disclosure further provides a pharmaceutical composition comprising the polynucleotides encoding the antibodies or antigen-binding fragments thereof, and one or more pharmaceutically acceptable carriers. Antibodies provided herein can also be produced in vivo by delivery of polynucleotides encoding the antibodies or antigen-binding fragments thereof provided herein, such as, for example, in-vitro-transcribed mRNA, or expression vectors. Methods are known in the art for polynucleotide delivery for antibody expression in vivo, see, for example, Rybakova, Y. et al, Molecular Therapy, vol. 27 (8), pp. 1415-1423 (2019); Deal, C. E. et al, Vaccines, 2021, 9, 108.

The present disclosure further provides pharmaceutical compositions comprising an expression vector comprising the polynucleotides encoding the antibodies or antigen-binding fragments thereof, and one or more pharmaceutically acceptable carriers.

In certain embodiments, the expression vector comprises a viral vector or a non-viral vector. Examples of viral vectors include, without limitation, adeno-associated virus (AAV) vector, lentivirus vector, retrovirus vector, and adenovirus vector. Examples of non-viral vectors include, without limitation, naked DNA, plasmid, exosome, mRNA, and so on. In certain embodiments, the expression vector is suitable for gene therapy in human. Suitable vectors for gene therapy include, for example, adeno-associated virus (AAV), or adenovirus vector. In certain embodiments, the expression vector comprises a DNA vector or a RNA vector. In certain embodiments, the pharmaceutically acceptable carriers are polymeric excipients, such as without limitation, microspheres, microcapsules, polymeric micelles and dendrimers. The polynucleotides, or polynucleotide vectors of the present disclosure may be encapsulated, adhered to, or coated on the polymer-based components by methods known in the art (see for example, W. Heiser, Nonviral gene transfer techniques, published by Humana Press, 2004; U.S. Pat. No. 6,025,337; Advanced Drug Delivery Reviews, 57 (15): 2177-2202 (2005)).

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising an antibody or antigen-binding fragment thereof and conjugates provided herein decreases oxidation of the antibody or antigen-binding fragment thereof. This reduction in oxidation prevents or reduces loss of binding affinity, thereby improving antibody stability and maximizing shelf-life. Therefore, in certain embodiments, pharmaceutical compositions are provided that comprise one or more antibodies or antigen-binding fragments thereof as disclosed herein and one or more antioxidants such as methionine. Further provided are methods for preventing oxidation of, extending the shelf-life of, and/or improving the efficacy of an antibody or antigen-binding fragment provided herein by mixing the antibody or antigen-binding fragment with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agent. The solvent may contain a buffer, such as citrate, sodium or potassium phosphate or other such buffer known to a person skilled in the art at, in one embodiment, about neutral pH. Subsequent sterile filtration of the solution followed by lyophilization under standard conditions known to a person skilled in the art provides a desirable formulation. In one embodiment, the resulting solution will be apportioned into vials for lyophilization. Each vial can contain a single dosage or multiple dosages of the antibody or antigen-binding fragment thereof or composition thereof. Overfilling vials with a small amount above that needed for a dose or set of doses (e.g. about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

The pharmaceutical composition described herein including pharmaceutically acceptable carriers such as addition salts or hydrates thereof, can be delivered to a patient using a wide variety of routes or modes of administration. Suitable routes of administration include, but inhalation, transdermal, oral, rectal, transmucosal, intestinal and parenteral administration, including intramuscular, subcutaneous and intravenous injections. Preferably, the pharmaceutical composition of the invention comprising an antibody or antibody fragment as the targeting moiety are administered parenterally, more preferably intravenously.

As used herein, the terms “administering” and “administration” are intended to encompass all means for directly and indirectly delivering the pharmaceutical composition to its intended site of action.

The pharmaceutical composition described herein, or pharmaceutically acceptable salts and/or hydrates thereof, may be administered singly, and/or in combination with other therapeutic agents. Of course, the choice of therapeutic agents that can be co-administered with the pharmaceutical composition of the disclosure will depend, in part, on the condition being treated.

For example, when administered to patients suffering from a disease state caused by an organism that relies on an autoinducer, the pharmaceutical composition of the disclosure can be administered in cocktails containing agents used to treat the pain, infection and other symptoms and side effects commonly associated with the disease. Such agents include, e.g., analgesics, antibiotics, etc.

When administered to a subject undergoing cancer treatment, the pharmaceutical composition may be administered in cocktails containing anti-cancer agents and/or supplementary potentiating agents. The pharmaceutical composition may also be administered in cocktails containing agents that treat the side-effects of radiation therapy, such as anti-emetics, radiation protectants, etc.

### V. Kits

In another aspect, the present disclosure provides a kit comprising the antibody or an antigen-binding fragment thereof provided herein.

In certain embodiments, the present disclosure provides a kit comprising the antibody or an antigen-binding fragment thereof provided herein, and a second therapeutic agent. In certain embodiments, the second therapeutic agent is selected from the group consisting of a chemotherapeutic agent, an anti-cancer drug, radiation therapy, an immunotherapy agent, an anti-angiogenesis agent, a targeted therapy, a cellular therapy, a gene therapy, a hormonal therapy, an antiviral agent, an antibiotic, an analgesics, an antioxidant, a metal chelator, and cytokines.

Such kits can further include, if desired, one or more of various conventional pharmaceutical kit components, such as, for example, containers with one or more pharmaceutically acceptable carriers, additional containers etc., as will be readily apparent to a person skilled in the art. Instructions, either as inserts or a labels, indicating quantities of the components to be administered, guidelines for administration, and/or guidelines for mixing the components, can also be included in the kit. The kit may also include one or more vial, test tube, flask, bottle, or syringe.

Other formats for kits will be apparent to those of skill in the art and are within the scope of the present disclosure.

### VI. Medical Use

### Therapeutic Use

In another aspect, the present disclosure provides a method for treating a disease or condition in a subject that is in need of such treatment, comprising: administering to the subject a pharmaceutical composition comprising a therapeutically effective amount of the antibody or antigen-binding fragments thereof of the present disclosure or a pharmaceutically acceptable salt thereof, and a pharmaceutical acceptable carrier.

The disease or condition need to be treated is related to the antigen(s) targeted by the antibody or antigen-binding fragments thereof. In some embodiments, the targeted antigen is selected from the group consisting of SIRPα, CLDN18.2, Siglec 15, HER2, EGFR, CD19, CD20, CD39, CD47, PD1, PDL1, CD3, NKG2D, NKG2A, Nkp46, CD137, OX40, CD40, etc.

In some embodiments, the uses of the pharmaceutical composition disclosed herein includes manufacturing of drugs for treating diseases such as cancer in a subject, such as a human being. By “cancer” herein is meant the pathological condition in humans that is characterized by unregulated cell proliferation.

In some embodiments, the disease is an SIRPα related disease, disorder or condition, which is characterized in expressing or over-expressing of SIRPα and/or SIRPα signature genes. In certain embodiments, the SIRPα related disease, disorder or condition include, but are not limited to, cancer, solid tumor, a chronic infection, an inflammatory disease, multiple sclerosis, an autoimmune disease, a neurologic disease, a brain injury, a nerve injury, a polycythemia, a hemochromatosis, a trauma, a septic shock, fibrosis, atherosclerosis, obesity, type II diabetes, a transplant dysfunction, or arthritis.

In some embodiments, the disease is an Claudin 18.2 related disease, disorder or condition, which is characterized in expressing or over-expressing of Claudin 18.2. Non-limiting examples of the Claudin 18.2 related disease include a cancer. In some embodiments, the cancer is an epithelial-cell derived cancer. In some embodiments, the cancer is anal cancer, appendix cancer, astrocytoma, basal cell carcinoma, gallbladder cancer, gastric cancer, lung cancer, bronchial cancer, bone cancer, liver and bile duct cancer, pancreatic cancer, breast cancer, liver cancer, ovarian cancer, testicle cancer, kidney cancer, renal pelvis and ureter cancer, salivary gland cancer, small intestine cancer, urethral cancer, bladder cancer, head and neck cancer, spine cancer, brain cancer, cervix cancer, uterine cancer, endometrial cancer, colon cancer, colorectal cancer, rectal cancer, esophageal cancer, gastrointestinal cancer, skin cancer, prostate cancer, pituitary cancer, vagina cancer, thyroid cancer, throat cancer, glioblastoma, melanoma, myelodysplastic syndrome, sarcoma, teratoma, chronic lymphocytic leukemia (CLL), chronic myeloid leukemia (CML), acute lymphocytic leukemia (ALL), acute myeloid leukemia (AML), Hodgkin lymphoma, non-Hodgkin lymphoma, multiple myeloma, T or B cell lymphoma, GI organ interstitialoma, soft tissue tumor, hepatocellular carcinoma, or adenocarcinoma, or the metastases thereof. In some embodiments, the cancer is gastric cancer, pancreatic cancer, esophagus cancer, ovarian cancer, or the metastases thereof.

The terms “treating” or “treatment” herein means therapeutic treatment and prophylactic or preventative treatment, wherein the objective is to reduce or prevent the aimed pathologic disorder or condition. In certain embodiments, “treatment” or “treating” includes (1) inhibiting a disease in a subject experiencing or displaying the pathology or symptoms of the disease, (2) ameliorating a disease in a subject that is experiencing or displaying the pathology or symptoms of the disease, and/or (3) affecting any measurable decrease in a disease in a subject or patient that is experiencing or displaying the pathology or symptoms of the disease.

The term “therapeutically effective amount” herein means an amount of the active ingredient (i.e., the antibody or antigen-binding fragments thereof) pharmaceutical composition provided herein effective to “treat” a disorder in a subject or mammal. Pharmaceutical compositions suitable for use with the present disclosure include compositions wherein the active ingredient is contained in a therapeutically effective amount, i.e., in an amount effective to achieve its intended purpose. The actual amount effective for a particular application will depend, inter alia, on the condition being treated. Determination of an effective amount is well within the capabilities of those skilled in the art, especially in light of the detailed disclosure herein.

In the case of cancer, the therapeutically effective amount of the drug may reduce the number of cancer cells, reduce the tumor size, inhibit cancer cell infiltration into peripheral organs, inhibit tumor metastasis, inhibit tumor growth to certain extent, and/or relieve one or more of the symptoms associated with the cancer to some extent.

In some embodiments, the antibodies or antigen-binding fragments thereof provided herein may be administered alone or in combination with a therapeutically effective amount of a second therapeutic agent. For example, the antibodies or antigen-binding fragments thereof disclosed herein may be administered in combination with a second therapeutic agent, for example, a chemotherapeutic agent, an anti-cancer drug, radiation therapy, an immunotherapy agent, an anti-angiogenesis agent, a targeted therapy, a cellular therapy, a gene therapy, a hormonal therapy, an antiviral agent, an antibiotic, an analgesics, an antioxidant, a metal chelator, or cytokines.

In certain of these embodiments, an antibody or antigen-binding fragment thereof provided herein that is administered in combination with one or more additional therapeutic agents may be administered simultaneously with the one or more additional therapeutic agents, and in certain of these embodiments the antibody or antigen-binding fragment thereof and the additional therapeutic agent(s) may be administered as part of the same pharmaceutical composition.

### Diagnostic Use

In another aspect, the present disclosure provides a method of diagnosing a disease, disorder or condition in a subject, comprising a) contacting a sample obtained from the subject with the antibody or antigen-binding fragments thereof provided herein; b) determining the presence or amount of the antigen targeted by the antibody or antigen-binding fragments thereof in the sample; and c) correlating the presence or the amount of the related antigen to existence or status of the disease, disorder or condition in the subject.

The term “diagnosis”, “diagnose” or “diagnosing” refers to the identification of a pathological state, disease or condition, such as identification of a specific antigen related disease, or refer to identification of a subject with the specific antigen related disease who may benefit from a particular treatment regimen. In some embodiments, diagnosis contains the identification of abnormal amount or activity of the specific antigen. In some embodiments, diagnosis refers to the identification of a cancer or an autoimmune disease in a subject.

As used herein, the term “sample” refers to a biological composition that is obtained or derived from a subject of interest that contains a cellular and/or other molecular entity that is to be characterized and/or identified, for example based on physical, biochemical, chemical and/or physiological characteristics. A sample includes, but is not limited to, cells, tissues, organs and/or biological fluids of a subject, obtained by any method known by those of skill in the art.

In another aspect, the present disclosure provides kits comprising the antibody or antigen-binding fragments thereof provided herein, optionally conjugated with a detectable moiety, which is useful in detecting a disease, disorder or condition. The kits may further comprise instructions for use.

The following examples are provided to better illustrate the claimed invention and are not to be interpreted as limiting the scope of the invention. All specific compositions, materials, and methods described below, in whole or in part, fall within the scope of the present disclosure. These specific compositions, materials, and methods are not intended to limit the invention, but merely to illustrate specific embodiments falling within the scope of the invention. A person skilled in the art may develop equivalent compositions, materials, and methods without the exercise of inventive capacity and without departing from the scope of the invention. It will be understood that many variations can be made in the procedures herein described while still remaining within the bounds of the present disclosure. It is the intention of the inventors that such variations are included within the scope of the invention.

All the numberings of the amino acid positions in the following examples were according to EU numbering system.

### EXAMPLES

### Example 1. Construction and Analysis of Asymmetric Monovalent Antibody with Shifted CH1-CL Interchain Disulfide Bond

Multiple potential sites for new disulfide bond formation on antibodies were identified. The potential sites were selected from amino acid residues in the CL region of the light chain and in the CH1 region of the heavy chain. A pair of non-cysteine residues, with one located in the CL region and the other located in the CH1 region were both replaced with cysteine in order to form a new disulfide bond between them. One or two such pairs of residues that could potentially form new interchain disulfide bonds were selected for each design and mutated to cysteines.

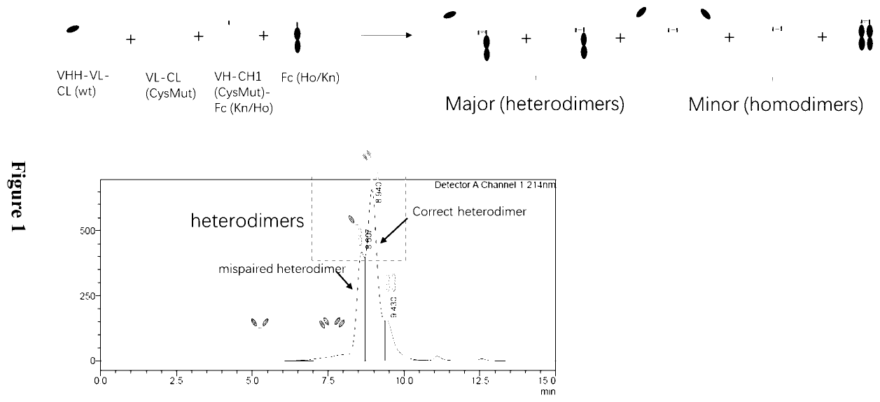

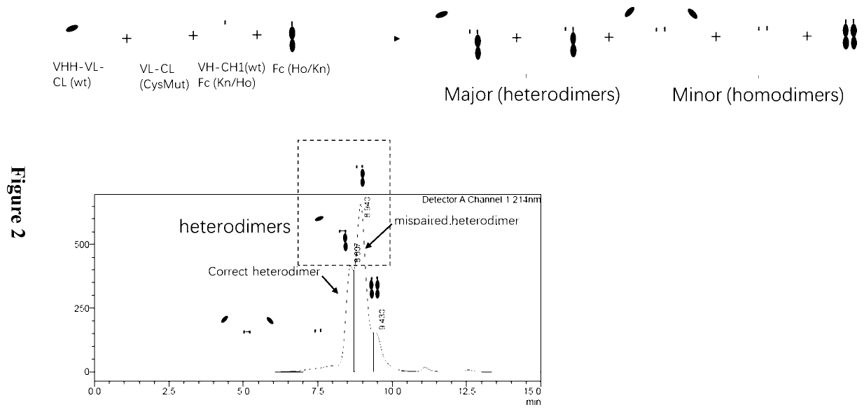

In addition, the conserved native disulfide bond between Cys220 in CH1 and Cys214 in CL were disrupted by replacing both residues with serine. For initial screening, an assay system with easy readout by SEC-HPLC was designed (FIG. 1 and FIG. 2).

Specifically, the system consists of 4 polypeptide chains. Chain 1 is a full-length HC with (Chain 1a used in screening assay 1, FIG. 1) or without (Chain 1b used in screening assay 2, FIG. 2) Cysteine mutations (from non-cysteine residue to cysteine residue) in the CH1 region and each chain 1 has Knob or Hole mutations in the Fc region. Chain 2 is a full-length LC with compatible Cysteine mutations in the CL region. Chain 3 is a wildtype LC with a single-domain antibody (VHH) fused at the N terminus. Chain 4 is an empty Fc with Hole or Knob mutations that are compatible with the Knob or Hole mutations in the full-length HC (i.e. Chain 1a or Chain 1b). With this system, the major products, which are monovalent heterodimers, consist of two possible molecular species that differ in sizes and are readily discernable and easily quantifiable in SEC-HPLC profile (FIG. 1 and FIG. 2). In both screening assays, heterodimers formed between mutant and wildtype peptide chains were considered mispaired heterodimers, whereas those between designed mutant chains or between wildtype chains were considered as correctly paired heterodimers. Hence, the relative abundance (RA) of different heterodimers, which was defined as

$$\frac{AUCcorrect heterodimer}{AUCcorrect heterodimer + AUCmispaired heterodimer} * 1 \; 00 \% ,$$

is a direct measurement of the pairing preference of different CL and CH1 chains.

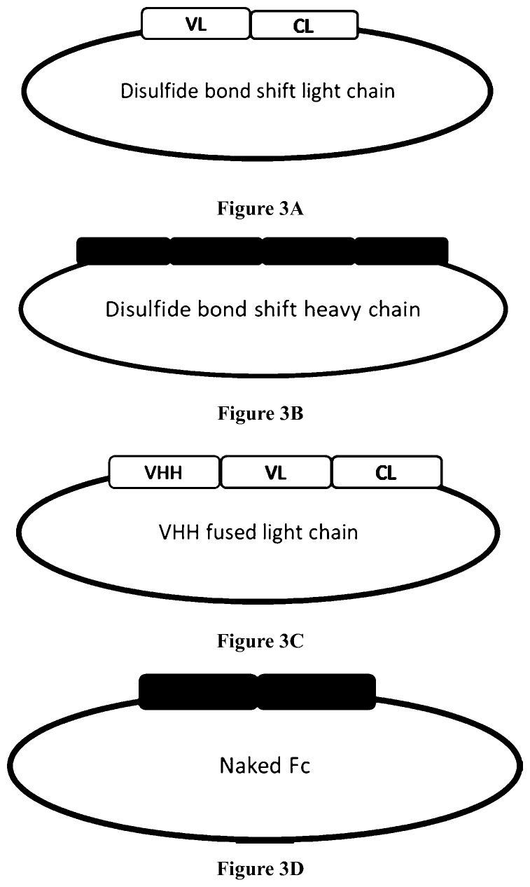

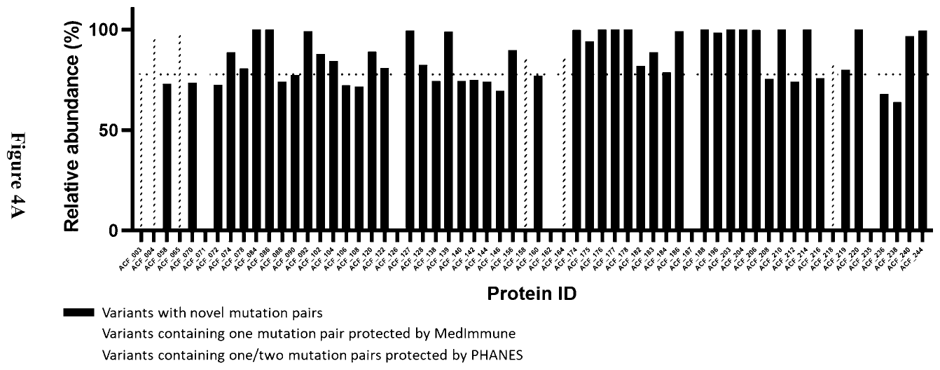

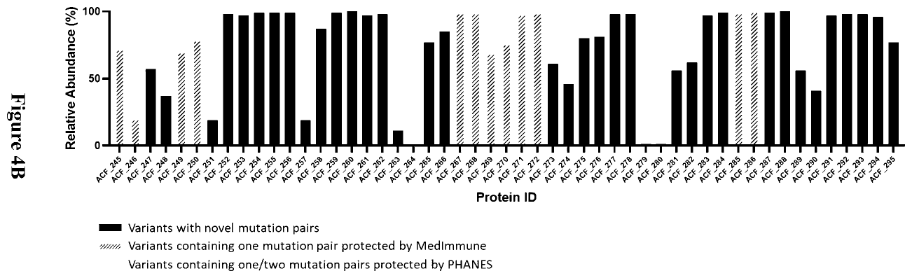

The sequences of different polypeptide chains of the two assay systems were listed in Table 6 below. The VH (SEQ ID NO: 3) and VL (SEQ ID NO: 2) sequences were derived from a humanized anti-CLDN18.2 antibody. Heavy chain constant regions including CH1 region (SEQ ID NO: 5) and Fc region (SEQ ID NO: 6) were derived from human IgG1. Light chain constant region CL (SEQ ID NO: 7) was derived from human kappa light chain. A VHH domain (SEQ ID NO: 1) was linked at the N-terminus of the VL region via a linker (SEQ ID NO: 9). All DNA fragments were synthesized by chemical process. Each of the four polypeptide chains was cloned into a pcDNA3.4 vector (FIGS. 3A to 3D). The four plasmids were mixed, and co-transfected into the ExpiCHO-S cells (Life Technologies). Then the expressed protein variants were produced in ExpiCHO-S cells using a 24-well plate production system. Culture supernatants were collected 7 days after transfection by centrifugation and incubated with protein A magnetic beads (GenScript). Bound antibodies were eluted by 0.05M citric buffer (pH 3.4) and neutralized immediately by Tris buffer (pH 9.0). Purified antibodies were evaluated by SDS-PAGE and SEC-HIPLC (Agilent).

TABLE 6
Amino acid sequences of the polypeptide chains for screening
assays 1 and 2

| Chain | Amino acid sequence | SEQ ID NO: |
|---|---|---|
| VHH | EVQVVESGGGLVQSGGSLRLSCAGSGFTESAGFM | 1 |
| (N- | VWHRQVPGKERELVALIATPSGSTQYADSVKGRF | |
| terminus | TISRDNGKNTVYLQMNSLKPEDTAVYYCNIRGYW | |
| part of | GQGTLVTVSS | |
| Chain 3) | | |
| VL (N- | DIVMTQSPDSLAVSLGERATINCKSSQSLLNAGNQ | 2 |
| terminus | KNYLTWYQQKPGQPPKLLIYWSSTRESGVPDRFS | |
| part of | GSGSGTDFTLTISSLQAEDVAVYHCONNYYYPLTF | |
| Chain | GGGTKLEIK | |
| 2) | | |
| VH (part | QVQLVQSGAEVKKPGASVKVSCKASGYTFTNWV | 3 |
| of Chain | HWVRQAPGQGLEWMGEINPTNARSNYNEKFKKR | |
| 1) | VTMTRDTSTSTVYMELSSLRSEDTAVYYCARIYY | |
| | GNSFAHWGQGTLVTVSS | |
| CL (part | RTVAAPSVFIFPPSDEQLKSGTASVVCLLNNFYPRE | 4 |
| of Chain | AKVQWKVDNALQSGNSQESVTEQDSKDSTYSLSS | |
| 3) | TLTLSKADYEKHKVYACEVTHQGLSSPVTKSFNR | |
| | GEC | |
| CH1 | ASTKGPSVFPLAPSSKSTSGGTAALGCLVKDYFPE | 5 |
| (part of | PVTVSWNSGALTSGVHTFPAVLQSSGLYSLSSVVT | |
| Chain 1) | VPSSSLGTQTYICNVNHKPSNTKVDKKVEPKSC | |
| Fc (wt) | DKTHTCPPCPAPELLGGPSVFLFPPKPKDTLMISRT | 6 |
| | PEVTCVVVDVSHEDPEVKFNWYVDGVEVHNAKT | |
| | KPREEQYNSTYRVVSVLTVLHQDWLNGKEYKCK | |
| | VSNKALPAPIEKTISKAKGQPREPQVYTLPPSRDEL | |
| | TKNQVSLTCLVKGFYPSDIAVEWESNGQPENNYK | |
| | TTPPVLDSDGSFFLYSKLTVDKSRWQQGNVFSCSV | |
| | MHEALHNHYTQKSLSLSPGK | |
| Fc | DKTHTCPPCPAPELLGGPSVFLFPPKPKDTLMISRT | 7 |
| (Hole) | PEVTCVVVDVSHEDPEVKFNWYVDGVEVHNAKT | |
| (Chain | KPREEQYNSTYRVVSVLTVLHQDWLNGKEYKCK | |
| 4a) | VSNKALPAPIEKTISKAKGQPREPQVYTLPPSRDEL | |
| | TKNQVSLSCAVKGFYPSDIAVEWESNGQPENNYK | |
| | TTPPVLDSDGSFFLVSKLTVDKSRWQQGNVFSCSV | |
| | MHEALHNHYTQKSLSLSPGK | |
| Fc | DKTHTCPPCPAPELLGGPSVFLFPPKPKDTLMISRT | 8 |
| (Knob) | PEVTCVVVDVSHEDPEVKFNWYVDGVEVHNAKT | |
| (Chain | KPREEQYNSTYRVVSVLTVLHQDWLNGKEYKCK | |
| 4b) | VSNKALPAPIEKTISKAKGQPREPQVYTLPPSRDEL | |
| | TKNQVSLWCLVKGFYPSDIAVEWESNGQPENNYK | |
| | TTPPVLDSDGSFFLYSKLTVDKSRWQQGNVFSCSV | |
| | MHEALHNHYTQKSLSLSPGK | |
| Linker | GGGGS | 9 |
| (between | | |
| VHH | | |
| and VL) | | |

One or two non-cysteine residues at various positions, including positions 114, 116, 118, 121, 124, 135, 137, 138, 160, 164 in the CL region of Chain 2 were mutated into cysteine, and one or two non-cysteine residues at various positions, including positions 126, 128, 129, 136, 141, 168, 170, 173, 175, 187 in the CH1 region of Chain 1 were mutated into cysteine. Multiple mutation pairs between Chain 1 and Chain 2 were formed using the mutation sites described above and used in screening assay 1.

In screening assay 1, wildtype and mutant CL were competing for mutant CH1. The correct pairing is formed between Chain 2 (with mutant CL) and Chain 1a (with mutant CH1) and mispairing is formed between Chain 3 (with wildtype CL) and Chain 1a (with mutant CH1).

In the screening assay 1, numerous mutation combinations of VL-CL (with mutations in the CL region) and VH—CH1-Fc (with mutations in CH1 region and Knob/Hole mutations in the Fc region) were tested. Table 7 and FIG. 4A shows the RA values of some mutant pairs that generated high RA values. ACF_003 is used as a benchmark in this screening assay 1. ACF_003 (including the mutation combination of S121C on the light chain and F126C on the heavy chain) has the corresponding mutations in CL region and CH1 region as V12 disclosed in U.S. Pat. No. 9,527,927.

TABLE 7
Mutations combinations showing high RA
values as tested in screening assay 1

| Protein ID | Mutant site in CL | Mutant site in CH1 |
|---|---|---|
| ACF_003 | S121C/C214S | F126C/C220S_hole |
| ACF_004 | S121C/C214S | F126C/C220S_Knob |
| ACF_058 | L135C/C214S | F170C/C220S_Hole |
| ACF_065 | S114C/S121C/C214S | S136C/F126C/C220S_Knob |
| ACF_070 | S114C/Q160C/C214S | S136C/Q175C/C220S_Hole |
| ACF_071 | S114C/Q160C/C214S | S136C/V173C/C220S_Knob |
| ACF_072 | S114C/Q160C/C214S | S136C/V173C/C220S_Hole |
| ACF_074 | S114C/T164C/C214S | S136C/F170C/C220S_Hole |
| ACF_078 | S114C/N137C/C214S | S136C/H168C/C220S_Hole |
| ACF_084 | F118C/S121C/C214S | L128C/F126C/C220S_Hole |
| ACF_086 | F118C/Q124C/C214S | L128C/F126C/C220S_Hole |
| ACF_088 | F118C/Q160C/C214S | L128C/Q175C/C220S_Hole |
| ACF_090 | F118C/Q160C/C214S | L128C/V173C/C220S_Hole |
| ACF_092 | F118C/T164C/C214S | L128C/F170C/C220S_Hole |
| ACF_102 | F118C/S121C/C214S | A129C/F126C/C220S_Hole |
| ACF_104 | F118C/Q124C/C214S | A129C/F126C/C220S_Hole |
| ACF_106 | F118C/Q160C/C214S | A129C/Q175C/C220S_Hole |
| ACF_108 | F118C/Q160C/C214S | A129C/V173C/C220S_Hole |
| ACF_120 | F118C/S121C/C214S | A141C/F126C/C220S_Hole |
| ACF_122 | F118C/Q124C/C214S | A141C/F126C/C220S_Hole |
| ACF_126 | F118C/Q160C/C214S | A141C/V173C/C220S_Hole |
| ACF_127 | F118C/T164C/C214S | A141C/F170C/C220S_Knob |
| ACF_128 | F118C/T164C/C214S | A141C/F170C/C220S_Hole |
| ACF_138 | F116C/S121C/C214S | S136C/F126C/C220S_Hole |
| ACF_139 | F116C/Q124C/C214S | S136C/F126C/C220S_Knob |
| ACF_140 | F116C/Q124C/C214S | S136C/F126C/C220S_Hole |
| ACF_142 | F116C/Q160C/C214S | S136C/Q175C/C220S_Hole |
| ACF_144 | F116C/Q160C/C214S | S136C/V173C/C220S_Hole |
| ACF_146 | F116C/T164C/C214S | S136C/F170C/C220S_Hole |
| ACF_156 | F116C/S121C/C214S | A141C/F126C/C220S_Hole |
| ACF_157 | F116C/Q124C/C214S | A141C/F126C/C220S_Knob |
| ACF_158 | F116C/Q124C/C214S | A141C/F126C/C220S_Hole |
| ACF_160 | F116C/Q160C/C214S | A141C/Q175C/C220S_Hole |
| ACF_162 | F116C/Q160C/C214S | A141C/V173C/C220S_Hole |
| ACF_164 | F116C/T164C/C214S | A141C/F170C/C220S_Hole |
| ACF_174 | Q124C/Q160C/C214S | F126/Q175C/C220S_Hole |
| ACF_175 | Q124C/Q160C/C214S | F126C/V173C/C220S_Knob |
| ACF_176 | Q124C/Q160C/C214S | F126C/V173C/C220S_Hole |
| ACF_177 | Q124C/T164C/C214S | F126C/F170C/C220S_Knob |
| ACF_178 | Q124C/T164C/C214S | F126C/F170C/C220S_Hole |
| ACF_182 | Q124C/N137C/C214S | F126C/H168C/C220S_Hole |
| ACF_183 | Q124C/N137C/C214S | F126C/T187C/C220S_Knob |
| ACF_184 | Q124C/N137C/C214S | F126C/T187C/C220S_Hole |
| ACF_186 | Q124C/N138C/C214S | F126C/H168C/C220S_Hole |
| ACF_187 | Q160C/L135C/C214S | V173C/F170C/C220S_Knob |
| ACF_188 | Q160C/L135C/C214S | V173C/F170C/C220S_Hole |
| ACF_196 | Q160C/L135C/C214S | Q175C/F170C/C220S_Hole |
| ACF_203 | Q160C/T164C/C214S | V173C/F170C/C220S_Knob |
| ACF_204 | Q160C/T164C/C214S | V173C/F170C/C220S_Hole |
| ACF_206 | Q160C/T164C/C214S | Q175C/F170C/C220S_Hole |
| ACF_208 | S114C/S162C/C214S | S136C/F170C/C220S_Hole |
| ACF_210 | F118C/S162C/C214S | L128C/F170C/C220S_Hole |
| ACF_212 | F118C/S162C/C214S | A129C/F170C/C220S_Hole |
| ACF_214 | F118C/S162C/C214S | A141C/F170C/C220S_Hole |
| ACF_216 | F116C/S162C/C214S | S136C/F170C/C220S_Hole |
| ACF_218 | F116C/S162C/C214S | A141C/F170C/C220S_Hole |
| ACF_219 | Q124C/S162C/C214S | F126C/F170C/C220S_Knob |
| ACF_220 | Q124C/S162C/C214S | F126C/F170C/C220S_Hole |
| ACF_235 | Q160C/L135C/C214S | V173C/V185C/C220S_Knob |
| ACF_236 | Q160C/L135C/C214S | V173C/V185C/C220S_Hole |
| ACF_238 | Q160C/L135C/C214S | Q175C/V185C/C220S_Hole |
| ACF_239 | N137C/S162C/C214S | F170C/T187C/C220S_Knob |
| ACF_240 | N137C/S162C/C214S | F170C/T187C/C220S_Hole |
| ACF_244 | N138C/S162C/C214S | H168C/F170C/C220S_Hole |

In screening assay 2, mutant and wildtype CL were competing for wildtype CH1. The RA value for mutant pairs used in the screening assay 2 were calculated by the same equation as in screening assay 1 except that the correct pairing is only formed between Chain 3 (with wildtype CL) and Chain 1b (with wildtype CH1) and mispairing is formed between Chain 2 (with mutant CL) and Chain 1b (with wildtype CH1).

Mutation pairs that generated high RA values were listed in Table 8. ACF_245 is used as a benchmark in this screening assay 2. ACF_245 (including the S121C mutation on the light chain and no mutation on the heavy chain) has the corresponding mutations in CL region as ACF_003 except for no mutation on the heavy chain. Table 8 and FIG. 4B shows the RA of some mutant pairs that generated high RA values.

TABLE 8
Mutations combinations showing high RA
values as tested in screening assay 2.

| Protein ID | Mutant sites in CL | Heavy chain |
|---|---|---|
| ACF_245 | S121C/C214S | wt-CH1_Fc_Hole |
| ACF_250 | S114C/S121C/C214S | wt-CH1_Fc_Knob |
| ACF_252 | S114C/Q160C/C214S | wt-CH1_Fc_Knob |
| ACF_253 | S114C/T164C/C214S | wt-CH1_Fc_Hole |
| ACF_254 | S114C/T164C/C214S | wt-CH1_Fc_Knob |
| ACF_255 | S114C/N137C/C214S | wt-CH1_Fc_Hole |
| ACF_256 | S114C/N137C/C214S | wt-CH1_Fc_Knob |
| ACF_258 | F118C/S121C/C214S | wt-CH1_Fc_Knob |
| ACF_259 | F118C/Q124C/C214S | wt-CH1_Fc_Hole |
| ACF_260 | F118C/Q124C/C214S | wt-CH1_Fc_Knob |
| ACF_261 | F118C/Q160C/C214S | wt-CH1_Fc_Hole |
| ACF_262 | F118C/Q160C/C214S | wt-CH1_Fc_Knob |
| ACF_265 | F116C/S121C/C214S | wt-CH1_Fc_Hole |
| ACF_266 | F116C/S121C/C214S | wt-CH1_Fc_Knob |
| ACF_267 | F116C/Q124C/C214S | wt-CH1_Fc_Hole |
| ACF_268 | F116C/Q124C/C214S | wt-CH1_Fc_Knob |
| ACF_270 | F116C/Q160C/C214S | wt-CH1_Fc_Knob |
| ACF_271 | F116C/T164C/C214S | wt-CH1_Fc_Hole |
| ACF_272 | F116C/T164C/C214S | wt-CH1_Fc_Knob |
| ACF_275 | Q124C/T164C/C214S | wt-CH1_Fc_Hole |
| ACF_276 | Q124C/T164C/C214S | wt-CH1_Fc_Knob |
| ACF_277 | Q124C/N138C/C214S | wt-CH1_Fc_Hole |
| ACF_278 | Q124C/N138C/C214S | wt-CH1_Fc_Knob |
| ACF_279 | Q160C/L135C/C214S | wt-CH1_Fc_Hole |
| ACF_280 | Q160C/L135C/C214S | wt-CH1_Fc_Knob |
| ACF_283 | F118C/S162C/C214S | wt-CH1_Fc_Hole |
| ACF_284 | F118C/S162C/C214S | wt-CH1_Fc_Knob |
| ACF_285 | F116C/S162C/C214S | wt-CH1_Fc_Hole |
| ACF_286 | F116C/S162C/C214S | wt-CH1_Fc_Knob |
| ACF_287 | N137C/S162C/C214S | wt-CH1_Fc_Hole |
| ACF_288 | N137C/S162C/C214S | wt-CH1_Fc_Knob |
| ACF_291 | Q124C/N137C/C214S | wt-CH1_Fc_Hole |
| ACF_292 | Q124C/N137C/C214S | wt-CH1_Fc_Knob |
| ACF_293 | S114C/S162C/C214S | wt-CH1_Fc_Hole |
| ACF_294 | S114C/S162C/C214S | wt-CH1_Fc_Knob |
| ACF_295 | Q124C/S162C/C214S | wt-CH1_Fc_Hole |
| ACF_296 | Q124C/S162C/C214S | wt-CH1_Fc_Knob |

### Example 2. Construction and Analysis of Symmetric Bivalent Monospecific Antibodies with Shifted CH1-CL Disulfide Bonds

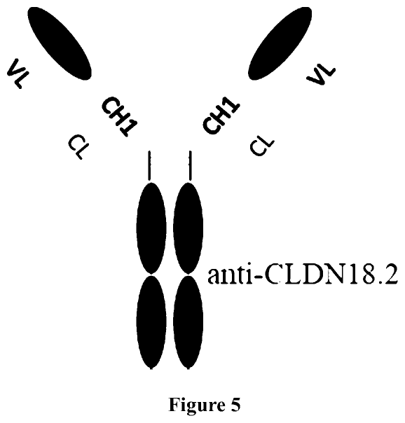

To further evaluate expression level and stability of the selected mutants in a reductionist system, some mutant pairs selected from the experiments of Example 1 were converted to symmetric bivalent monospecific antibodies (FIG. 5, Table 9) for the yield and purity test.

All the mutations were made based on the parent antibody hu28. The amino acid sequences of VL and VH regions of hu28 were as shown in SEQ ID NOs: 2 and 3, respectively. The amino acid sequence of CL region of hu28 was as shown in SED ID NO: 4. The amino acid sequence of CH1 region of hu28 was as shown in SED ID NO: 5. The amino acid sequence of Fc region of hu28 was as shown in SED ID NO: 6.

TABLE 9
Mutation combinations in the bivalent
monospecific antibodies for test.

| Protein ID | Mutant site in CL | Mutant site in CH1 |
|---|---|---|
| ACF-hu28_001 | S121C/C214S | F126C/C220S |
| ACF-hu28_002 | L135C/C214S | F170C/C220S |
| ACF-hu28_003 | S114C/S121C/C214S | S136C/F126C/C220S |
| ACF-hu28_004 | S114C/Q160C/C214S | S136C/V173C/C220S |
| ACF-hu28_005 | S114C/T164C/C214S | S136C/F170C/C220S |
| ACF-hu28_006 | S114C/N137C/C214S | S136C/H168C/C220S |
| ACF-hu28_007 | F118C/S121C/C214S | L128C/F126C/C220S |
| ACF-hu28_008 | F118C/S121C/C214S | A129C/F126C/C220S |
| ACF-hu28_009 | F118C/S121C/C214S | A141C/F126C/C220S |
| ACF-hu28_010 | F118C/Q124C/C214S | L128C/F126C/C220S |
| ACF-hu28_011 | F118C/Q124C/C214S | A129C/F126C/C220S |
| ACF-hu28_012 | F118C/Q124C/C214S | A141C/F126C/C220S |
| ACF-hu28_013 | F118C/Q160C/C214S | A141C/Q175C/C220S |
| ACF-hu28_014 | F118C/Q160C/C214S | A141C/V173C/C220S |
| ACF-hu28_015 | F118C/T164C/C214S | L128C/F170C/C220S |
| ACF-hu28_016 | F118C/T164C/C214S | A141C/F170C/C220S |
| ACF-hu28_017 | F116C/S121C/C214S | A141C/F126C/C220S |
| ACF-hu28_018 | F116C/Q124C/C214S | S136C/F126C/C220S |
| ACF-hu28_019 | F116C/Q124C/C214S | A141C/F126C/C220S |
| ACF-hu28_020 | F116C/Q160C/C214S | A141C/V173C/C220S |
| ACF-hu28_021 | F116C/Q160C/C214S | A141C/Q175C/C220S |
| ACF-hu28_022 | F116C/T164C/C214S | A141C/F170C/C220S |
| ACF-hu28_023 | Q124C/Q160C/C214S | F126C/Q175C/C220S |
| ACF-hu28_024 | Q124C/Q160C/C214S | F126C/V173C/C220S |
| ACF-hu28_025 | Q124C/T164C/C214S | F126C/F170C/C220S |
| ACF-hu28_026 | Q124C/N137C/C214S | F126C/H168C/C220S |
| ACF-hu28_027 | Q124C/N137C/C214S | F126C/T187C/C220S |
| ACF-hu28_028 | Q124C/N138C/C214S | F126C/H168C/C220S |
| ACF-hu28_029 | Q160C/L135C/C214S | V173C/F170C/C220S |
| ACF-hu28_030 | Q160C/L135C/C214S | Q175C/F170C/C220S |
| ACF-hu28_031 | Q160C/L135C/C214S | V173C/V185C/C220S |
| ACF-hu28_032 | Q160C/T164C/C214S | V173C/F170C/C220S |
| ACF-hu28_033 | Q160C/T164C/C214S | Q175C/F170C/C220S |
| ACF-hu28_034 | S114C/S162C/C214S | S136C/F170C/C220S |
| ACF-hu28_035 | F118C/S162C/C214S | L128C/F170C/C220S |
| ACF-hu28_036 | F118C/S162C/C214S | A141C/F170C/C220S |
| ACF-hu28_037 | F116C/S162C/C214S | A141C/F170C/C220S |
| ACF-hu28_038 | Q124C/S162C/C214S | F126C/F170C/C220S |
| ACF-hu28_039 | N137C/S162C/C214S | F170C/T187C/C220S |
| ACF-hu28_040 | N138C/S162C/C214S | H168C/F170C/C220S |

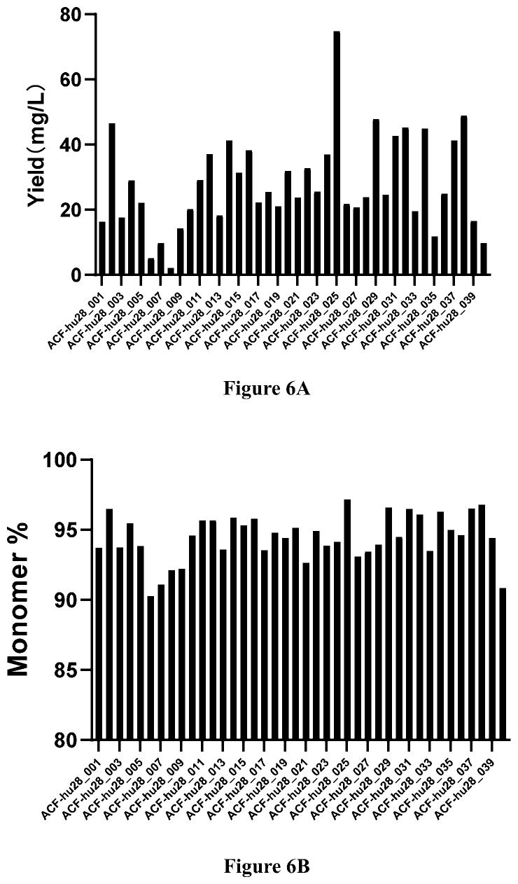

All DNA fragments were synthesized and cloned into pcDNA3.4 vectors. LC and HC vectors containing selected residue substitution pairs were co-transfected into ExpiCHO-S cells using a 24-well plate production system and purified by protein A beads. Purified antibodies were first evaluated by SDS-PAGE and HPLC-SEC for purity. Yield and purity of each antibody were shown in FIGS. 6A and 6B, respectively. ACF-hu28_001 containing residue substitution pairs reported by MedImmune in U.S. Pat. No. 9,527,927 was used as a reference for comparison.

As shown in the results in FIGS. 6A and 6B, ACF-hu28_025, ACF-hu28_038, ACF-hu28_029, ACF-hu28_037, ACF-hu28_002, ACF-hu28_031, ACF-hu28_034, ACF-hu28_032, ACF-hu28_014, ACF-hu28_016, ACF-hu28_012, ACF-hu28_011, ACF-hu28_004, ACF-hu28_015, ACF-hu28_020, ACF-hu28_035, ACF-hu28_022, ACF-hu28_018, ACF-hu28_036, ACF-hu28_010, ACF-hu28_030, ACF-hu28_039, ACF-hu28_019, ACF-hu28_024, ACF-hu28_028, ACF-hu28_023, ACF-hu28_005, ACF-hu28_003 have comparable or higher production yield and monomer percentage and as compared to the reference monoclonal antibody ACF hu28_001 (S121C/C214S, F126C/C220S).

### Example 3. Antigen Binding Measurement and Thermostability Comparison of Bivalent Monospecific Antibodies

### 3.1 Binding Activity Evaluation by FACS Assay

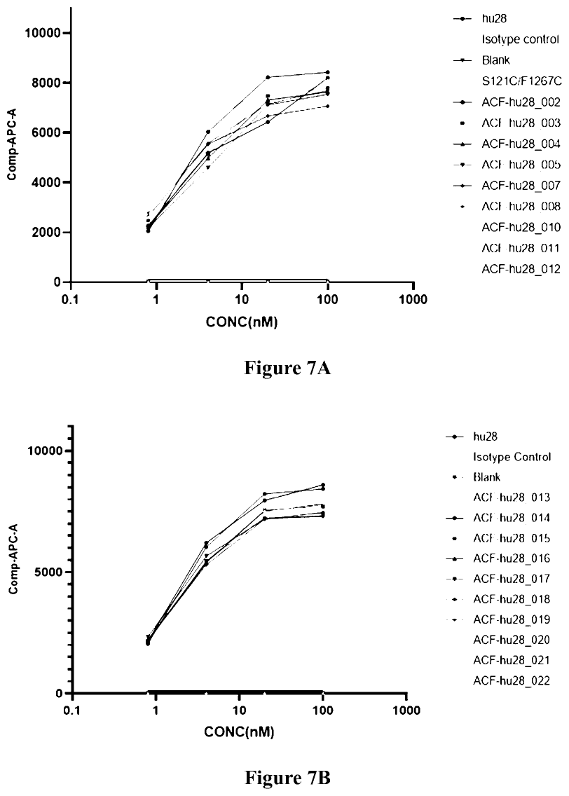

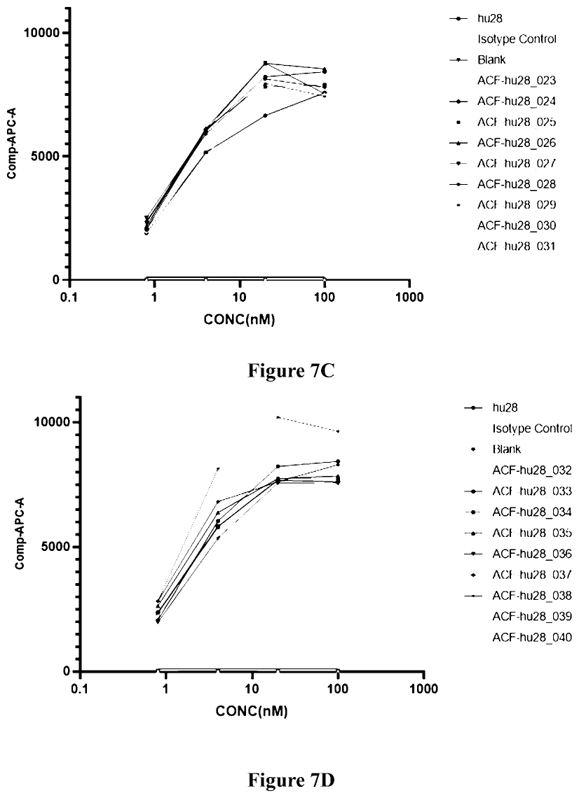

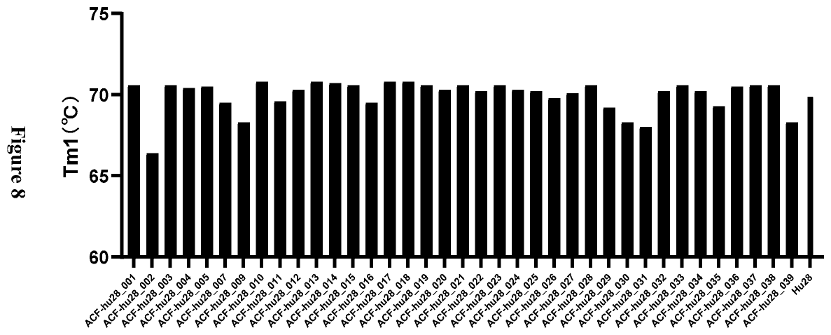

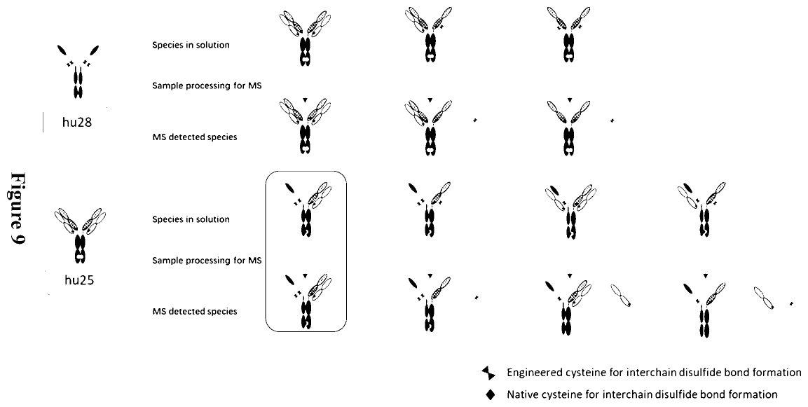

All selected bivalent variants were compared for binding to CLDN18.2 by FACS using CLDN18.2-overexpressing MC38 cells (FIGS. 7A to 7D). All proteins were buffer-exchanged into PBS (pH7.2) and diluted into different concentrations (100 nM, 20 nM, 4 nM, 0.8 nM) with FACS buffer (BD). Hu28 mAb were used as positive controls.

No significant difference of antigen binding activity was observed between the variants and positive control mAb.

3.2 Themo Stability Analysis of Mutant mAbs

Thermostability of the purified variants were assessed using thermal shift assay (FIG. 8). The antibody variants were first buffer-exchanged into PBS (pH 7.2), before mixing with freshly diluted Protein Thermal Shift Dye (Thermo Fisher Scientific). The mixtures were then transferred into a 384-well plate and loaded onto the QuantStudio Real-Time PCR system (Thermo Fisher Scientific) for Tm measurement. Temperature was scanned from 25° C. to 99° C. with a ramp rate of 1.6° C./sec and a 2-min hold time. The melting curves obtained were analyzed with the Protein Thermal Shift Software. The data suggested that all variants have comparable thermostability to the wildtype monoclonal antibody.

### Example 4. Asymmetric Bispecific Antibody Generation and Analysis by LC-MS

In order to quantitatively evaluate the efficiency of cognate CH1 and CL chain pairing in a bispecific context, two series of bispecific antibody were generated and analyzed with LC-MS.

In both series, one arm of the bispecific antibodies was derived from the anti-CLDN 18.2 antibody (hu28) and the other arm was derived from the anti-SIRPα antibody (hu25). Knob mutation was introduced into the CH3 region of the hu28 arm and hole mutations were introduced into the CH3 region of the hu25 arm. In addition, mutations that abolish protein A binding were introduced into the hu25 arm to simplify the system.

The amino acid sequences of V_{H} and V_{L} regions of hu25 were as shown in SEQ ID NOs: 10 and 11, respectively (as shown in Table 10). The amino acid sequences of V_{H} and V_{L} regions of hu28 were as shown in SEQ ID NOs: 2 and 3, respectively. The amino acid sequence of non-mutated CL region of hu25 or hu28 was as shown in SEQ ID NO: 4. The amino acid sequence of non-mutated CH1 region of hu25 or hu28 was as shown in SEQ ID NO: 5. The amino acid sequence of Fc (with “Hole” mutations) of hu25 or hu28 was as shown in SEQ ID NO: 7. The amino acid sequence of Fc (with “Knob” mutations) of hu25 or hu28 was as shown in SEQ ID NO: 8.

TABLE 10
Amino acid sequences of hu25.

| | | SEQ |
|---|---|---|
| | | ID |
| Chain | Amino acid sequence | NO: |
| hu25 | EVQLVQSGAEVKKPGATVKISCKVSGFNIKDY | 10 |
| VH | YMHWVRQAPGKGLEWIGRVDPEDAETKYAEKF | |
| | QGRVTITADTSTDTAYMELSSLRSEDTAVYYC | |
| | NRGLVYWGQGTLVTVSS | |
| hu25 | EIVLTQSPATLSLSPGERATLSCRASQSVSSS | 11 |
| VL | YLYWYQQKPGQAPKLLIYSASNRASGIPARFS | |
| | GSGSGTDYTLTISSLEPEDFAVYYCHQWSSYP | |
| | YTFGQGTKLEIK | |

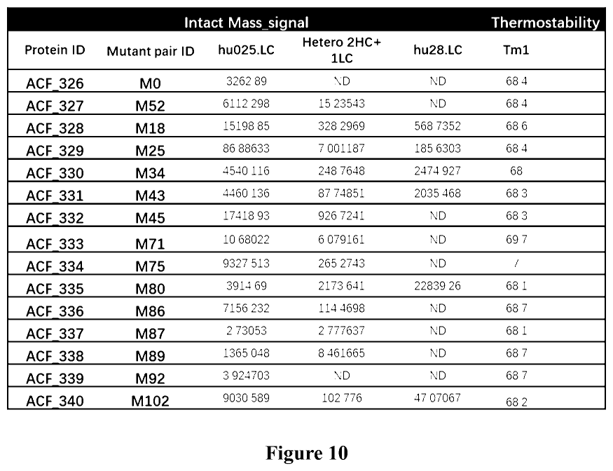

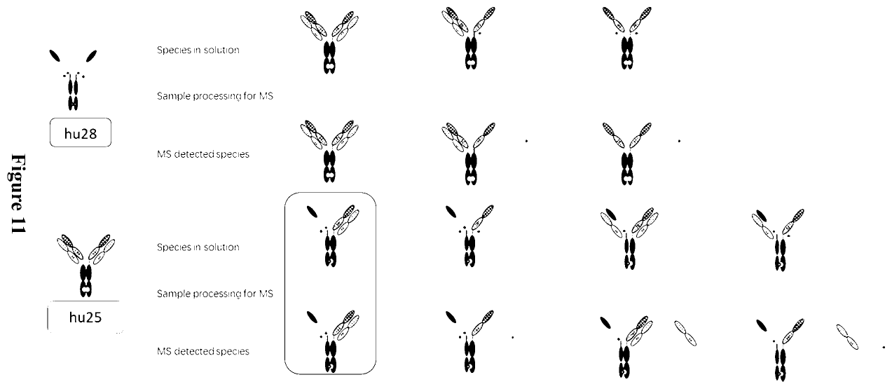

In Case 1 study, different mutation pairs for shifted disulfide bond formation were introduced into the CH1 and CL regions of the hu28 arm and wildtype sequences were retained in the hu25 arm (FIGS. 9 and 10, Table 11).

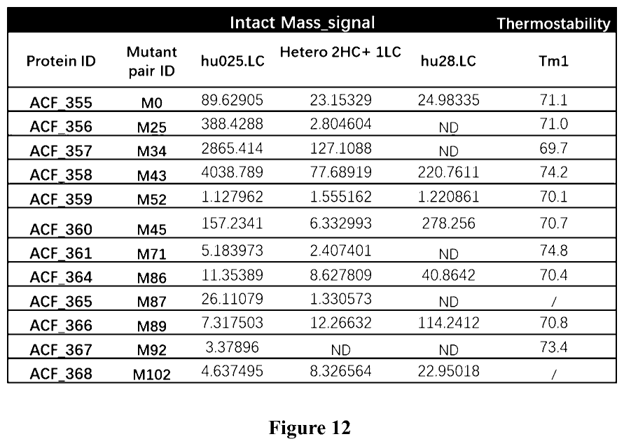

In Case 2 study, the same set of mutation pairs were introduced into the hu25 arm and wildtype sequences were retained in the hu28 arm (FIGS. 11 and 12, Table 11).

TABLE 11
Different construct designs in Cases 1 and 2

| | |
|---|---|
| Case 1 | Case 2 |

| | hu28 | hu25 | hu28 | hu25 |
|---|---|---|---|---|
| Knob/Hole | Knob | Hole | Knob | Hole |
| RF mutation | Yes | No | Yes | No |
| CH1-CL | Mutant | Wildtype | Wildtype | Mutant |

All the mutation combinations constructed for test were shown in Table 12.

TABLE 12
Mutation combinations in the Case
1 study and Case 2 study for test.

| Protein ID | Mutant site in CL | Mutant site in CH1 |
|---|---|---|
| ACF_326 | S121C/C214S | F126C/C220S_Knob |
| ACF_327 | F116C/Q124C/C214S | S136C/F126C/C220S_Knob |
| ACF_328 | S114C/Q160C/C214S | S136C/V173C/C220S_Knob |
| ACF_329 | F118C/Q124C/C214S | L128C/F126C/C220S_Knob |
| ACF_330 | F118C/Q124C/C214S | A129C/F126C/C220S_Knob |
| ACF_331 | F118C/Q124C/C214S | A141C/F126C/C220S_Knob |
| ACF_332 | F118C/Q160C/C214S | A141C/V173C/C220S_Knob |
| ACF_333 | Q124C/T164C/C214S | F126C/F170C/C220S_Knob |
| ACF_334 | Q124C/N138C/C214S | F126C/H168C/C220S_Knob |
| ACF_335 | Q160C/L135C/C214S | Q175C/F170C/C220S_Knob |
| ACF_336 | S114C/S162C/C214S | S136C/F170C/C220S_Knob |
| ACF_337 | F118C/S162C/C214S | L128C/F170C/C220S_Knob |
| ACF_338 | F118C/S162C/C214S | A141C/F170C/C220S_Knob |
| ACF_339 | Q124C/S162C/C214S | F126C/F170C/C220S_Knob |
| ACF_340 | N137C/S162C/C214S | F170C/T187C/C220S_Knob |
| ACF_355 | S121C/C214S | F126C/C220S_hole |
| ACF_356 | F118C/Q124C/C214S | L128C/F126C/C220S_Hole |
| ACF_357 | F118C/Q124C/C214S | A129C/F126C/C220S_Hole |
| ACF_358 | F118C/Q124C/C214S | A141C/F126C/C220S_Hole |
| ACF_359 | F116C/Q124C/C214S | S136C/F126C/C220S_Hole |
| ACF_360 | F118C/Q160C/C214S | A141C/V173C/C220S_Hole |
| ACF_361 | Q124C/T164C/C214S | F126C/F170C/C220S_Hole |
| ACF_364 | S114C/S162C/C214S | S136C/F170C/C220S_Hole |
| ACF_365 | F118C/S162C/C214S | L128C/F170C/C220S_Hole |
| ACF_366 | F118C/S162C/C214S | A141C/F170C/C220S_Hole |
| ACF_367 | Q124C/S162C/C214S | F126C/F170C/C220S_Hole |
| ACF_368 | N137C/S162C/C214S | F170C/T187C/C220S_Hole |

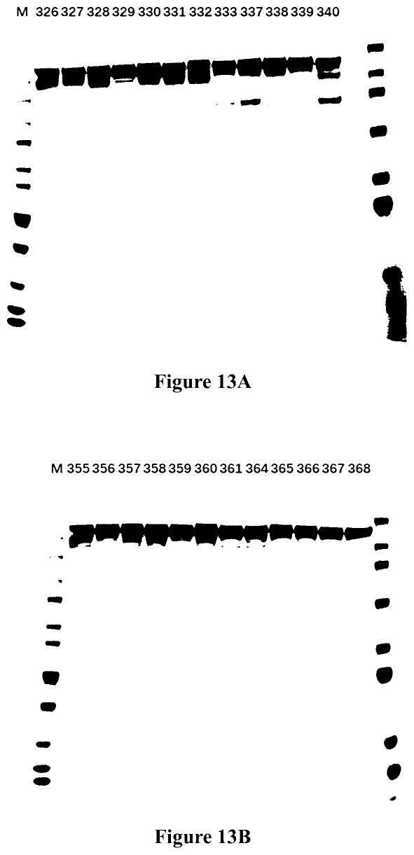

To produce the bispecific antibodies, plasmids of hu28 HC and LC were mixed with plasmids of hu25 HC and LC prior to co-transfection. The variants were produced in ExpiCHO-S cells (Life Technologies) using a 24-well plate production system. Culture supernatants were collected 7 days after transfection by centrifugation and incubated with protein A magnetic beads (GenScript). Bound antibodies were eluted by 0.05M citric buffer (pH 3.4) and neutralized immediately by Tris buffer (pH 9.0). The purified antibodies were analyzed first with SDS-PAGE (FIGS. 13A and 13B).

In order to quantitatively evaluate the efficiency of cognate CH1 and CL chain pairing, the purified antibodies were analyzed by LC-MS under non-reducing condition. Due to denaturing nature of sample processing conditions in LC-MS analysis, the molecules containing mispairing HC-LC (CH1 mutated HC paired with wildtype LC or CH1 unmodified HC paired with CL mutated LC) will be dissociated and show LC only peaks in LC-MS profile. Thus, in theory, all the molecules containing heterodimeric HC will be detected. Heterodimeric HCs with two correctly paired LCs were desired bispecific intact antibody. Heterodimeric HCs with mis-paired LC would be detected by the mis-paired and dissociated LC peaks in LC-MS profile. By assessing the intensity of MS signals for LC only peaks, which are derived from CH1-CL mispairing, it is possible to rank the cognate CH1 and CL chain pairing efficiency of different mutation pairs (FIGS. 9 and 11).

Thermostability of the antibodies were evaluated by differential scanning fluorimetry.

In Case 1 study, ACF_329, 333, 337, 338 and 339 exhibited lower signals of hu025 LC and equal signal of hu028 LC than the benchmark ACF_326 (MedImmune design) (FIG. 10). These data indicated that mutation pairs introduced in ACF_329, 333, 337, 338 and 339 provided higher efficiency of cognate CH1 and CL chain pairing in this case.

In Case 2 study, ACF_356, 359, 361, 364, 365, 366, 367, 368 exhibited comparable signals of hu025 LC and hu028 LC to the benchmark ACF_355 (MedImmune design) (FIG. 12). These data indicated that mutation pairs introduced in ACF_356, 359, 361, 364, 365, 366, 367 and 368 provided high efficiency of cognate CH1 and CL chain pairing.

It's notable that mutation pairs as listed below provided high efficiency of cognate CH1 and CL chain pairing in both cases: (i) F118C/Q124C on light chain and L128C/F126C on heavy chain; (ii) Q124C/T164C on light chain and F126C/F170C on heavy chain; (iii) F118C/S162C on light chain and L128C/F170C on heavy chain; (iv) F118C/S162C on light chain and A141C/F170C on heavy chain; and (v) Q124C/S162C on light chain and F126C/F170C on heavy chain.

### Example 5. Further Generation and Analysis of Asymmetric Bispecific Antibodies

### 5.1 Protein Expression and Purification

### 5.1.1 Constructs and Amino Acid Sequences of the Tested Antibodies

Three bispecific antibodies were expressed and purified, namely Antibody 1, Antibody 2, and Antibody 3.

In Antibody 1 study, the mutation pairs of Q124C/T164C/C214S on light chain and F126C/F170C/C220S on heavy chain were introduced into the hu25 arm and wildtype sequences were retained in the hu28 arm. Antibody 1 has the same structure and amino acid sequences as those of antibody ACF_361 in Example 4. Therefore, it is indicated as ACF_361 or ACF361 hereinafter.

In Antibody 2 study, the mutation pairs of Q124C/T164C/C214S on light chain and F126C/F170C/C220S on heavy chain were introduced into the hu25 arm and wildtype sequences were retained in the hu29 (source sequence: Atezolizumab, an anti-PDL1 antibody) arm. Antibody 2 is indicated as ACF_389 or ACF389 hereinafter.

The amino acid sequences of VH and VL regions of hu25 were as shown in SEQ ID NOs: 10 and 11, respectively (Table 10). The amino acid sequences of VH and VL regions of hu29 were as shown in SEQ ID NOs: 12 and 13, respectively (Table 11). The amino acid sequence of non-mutated CL region of hu25 or hu29 was as shown in SEQ ID NO: 4. The amino acid sequence of non-mutated CH1 region of hu25 or hu29 was as shown in SEQ ID NO: 5. The amino acid sequence of Fc (with “Hole” mutations) of hu25 or hu29 was as shown in SEQ ID NO: 7. The amino acid sequence of Fc (with “Knob” mutations) of hu25 or hu29 was as shown in SEQ ID NO: 8.

TABLE 11
Amino acid sequences of heavy chain and light
chain variable regions of hu29

| | | SEQ |
|---|---|---|
| | | ID |
| Chain | Amino acid sequence | NO: |
| hu29 | EVQLVESGGGLVQPGGSLRLSCAASGFTFSDSW | 12 |
| VH | IHWVRQAPGKGLEWVAWISPYGGSTYYADSVKG | |
| | RFTISADTSKNTAYLQMNSLRAEDTAVYYCARR | |
| | HWPGGFDYWGQGTLVTVSS | |
| hu29 | DIQMTQSPSSLSASVGDRVTITCRASQDVSTAV | 13 |
| VL | AWYQQKPGKAPKLLIYSASFLYSGVPSRFSGSG | |
| | SGTDFTLTISSLQPEDFATYYCQQYLYHPATFG | |
| | QGTKVEIK | |

In Antibody 3 study, the mutation pairs of Q124C/S162C/C214S on light chain and F126C/F170C/C220S on heavy chain were introduced into the H95 arm and wildtype sequences were retained in the H43 arm. Antibody 3 was indicated as ESB07.451 hereinafter.

The amino acid sequences of V_{H} and V_{L} regions of H95 were as shown in SEQ ID NOs: 14 and 15, respectively. The amino acid sequences of VH and VL regions of H43 were as shown in SEQ ID NOs: 16 and 17, respectively. The amino acid sequence of non-mutated CL region of H95 or H43 was as shown in SEQ ID NO: 19. The amino acid sequence of non-mutated CH1 region of H95 or H43 was as shown in SEQ ID NO: 18. The amino acid sequence of Fc (with “Hole” mutations) of H43 was as shown in SEQ ID NO: 21. The amino acid sequence of Fc (with “Knob” mutations) of H95 was as shown in SEQ ID NO: 20.

TABLE 12
Amino acid sequences of H95 and H43

| Chain | Amino acid sequence | SEQ ID NO: |
|---|---|---|
| H95 VH | QVQLVQSGAEVKKPGASVKVSCKASGYTFTDYYI | 14 |
| | NWVRQAPGQRLEWMGWIYPGSGNTKYNEKFKG | |
| | RVTITRDTSASTAYMELSSLRSEDTAVYYCARGLL | |
| | GPGDYWGQGTTVTVSS | |
| H95 VL | DIVMTQSPDSLAVSLGERATINCKASQSVSNDVA | 15 |
| | WYQQKPGQPPKLLIYYASNRYTGVPDRFSGSGSG | |
| | TDFTLTISSLQAEDVAVYYCQQDYSSPYTFGGGTK | |
| | LEIK | |
| H43 VH | QVQLVQSGAEVKKPGASVKVSCKASGYTFTSYW | 16 |
| | MYWVRQAPGQGLEWIGHINPSSGYTKYNQKFKD | |
| | KATLTADKSTSTAYMELSSLRSEDTAVYYCARDP | |
| | NWDNYWGQGTTVTVSS | |
| H43 VL | DIQMTQSPSSMSASVGDRVTITCKASQDVSIAVAW | 17 |
| | YQQKPGKAPKLLIYSASYRYTGVPSRFSGSGSGTD | |
| | FTFTISSVQPEDIATYYCQQHYSTPYTFGGGTKLEI | |
| | K | |
| H95/H43 | ASTKGPSVCPLAPCSRSTSESTAALGCLVKDYFPEP | 18 |
| CH1 | VTVSWNSGALTSGVHTCPAVLQSSGLYSLSSVVT | |
| | VPSSSLGTKTYTCNVDHKPSNTKVDKKVEPKSC | |
| H95/H43 | RTVAAPSVFIFPPSDECLKSGTASVVCLLNNFYPRE | 19 |
| CL | AKVQWKVDNALQSGNSQECVTEQDSKDSTYSLSS | |
| | TLTLSKADYEKHKVYACEVTHQGLSSPVTKSFNR | |
| | GES | |
| H95 Fc | DKTHTCPPCPAPELLGGPSVFLFPPKPKDTLMISRT | 20 |
| (Knob) | PEVTCVVVDVSHEDPEVKFNWYVDGVEVHNAKT | |
| | KPREEQYNSTYRVVSVLTVLHQDWLNGKEYKCK | |
| | VSNKALPAPIEKTISKAKGQPREPQVYTLPPSRDEL | |
| | TKNQVSLWCLVKGFYPSDIAVEWESNGQPENNYK | |
| | TTPPVLDSDGSFFLYSKLTVDKSRWQQGNVFSCSV | |
| | MHEALHNHYTQKSLSLSPGK | |
| H43 Fc | DKTHTCPPCPAPELLGGPSVFLFPPKPKDTLMISRT | 21 |
| (Hole) | PEVTCVVVDVSHEDPEVKFNWYVDGVEVHNAKT | |
| | KPREEQYNSTYRVVSVLTVLHQDWLNGKEYKCK | |
| | VSNKALPAPIEKTISKAKGQPREPQVYTLPPSRDEL | |
| | TKNQVSLSCAVKGFYPSDIAVEWESNGQPENNYK | |
| | TTPPVLDSDGSFFLVSKLTVDKSRWQQGNVFSCSV | |
| | MHEALHNRFTQKSLSLSPGK | |

### 5.1.2 Expression and Purification of the Tested Antibodies

To produce the bispecific antibodies, prior to co-transfection, plasmids of hu28 HC & LC were mixed with plasmids of hu25 HC & LC, plasmids of hu29 HC & LC were mixed with plasmids of hu25 HC & LC, and plasmids of H95 HC and LC were mixed with plasmids of H43 HC and LC.

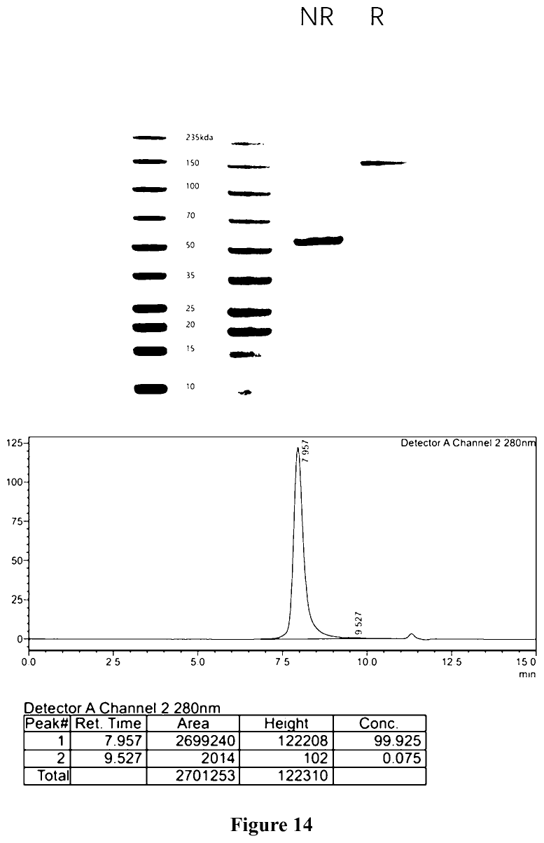

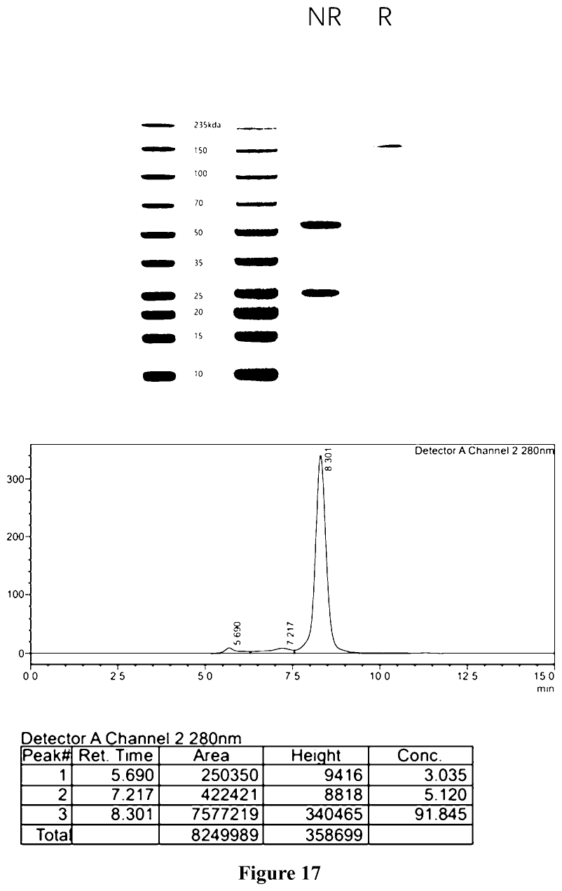

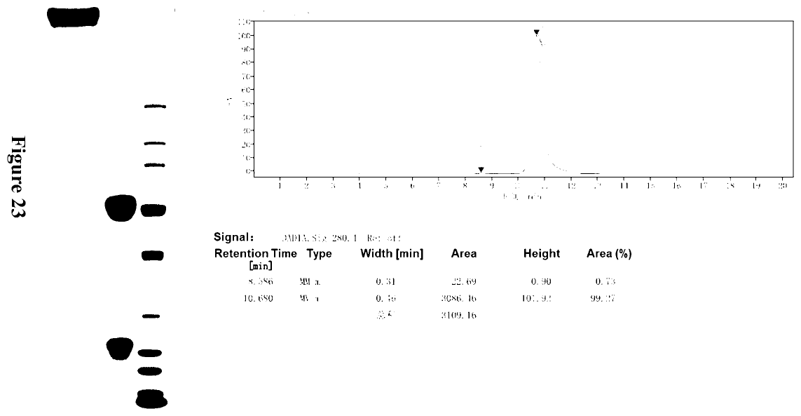

The variants were produced in ExpiCHO-S cells (Life Technologies). Culture supernatants were collected 7 days after transfection by centrifugation and incubated with alkali-resistant Protein A medium (AT Protein A Diamond, bestchrom). Bound antibodies were eluted by 0.05M citric buffer (pH 3.4) and neutralized immediately by Tris buffer (pH 9.0). The purified antibodies were analyzed first with SDS-PAGE and SEC. The results for ACF_361, ACF_389 and ESB07.451 are shown in FIG. 14, FIG. 17 and FIG. 22, respectively.

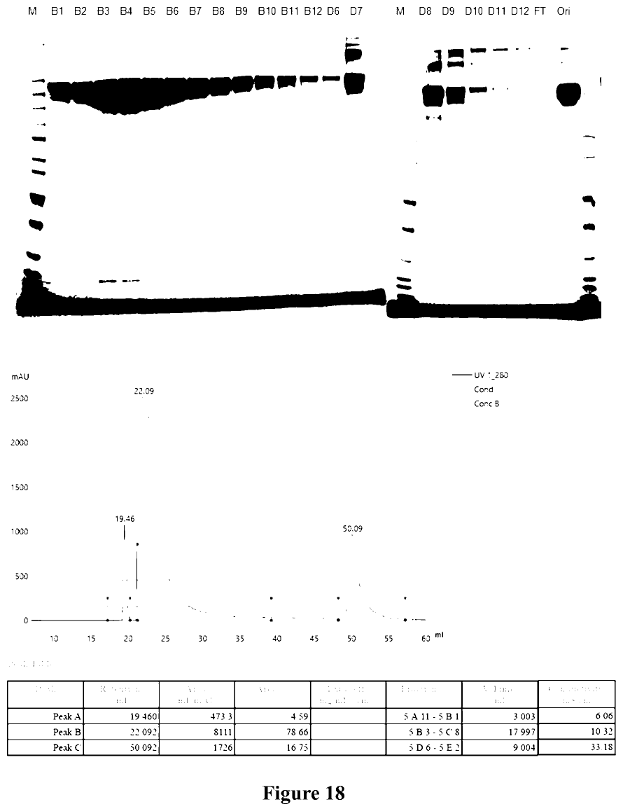

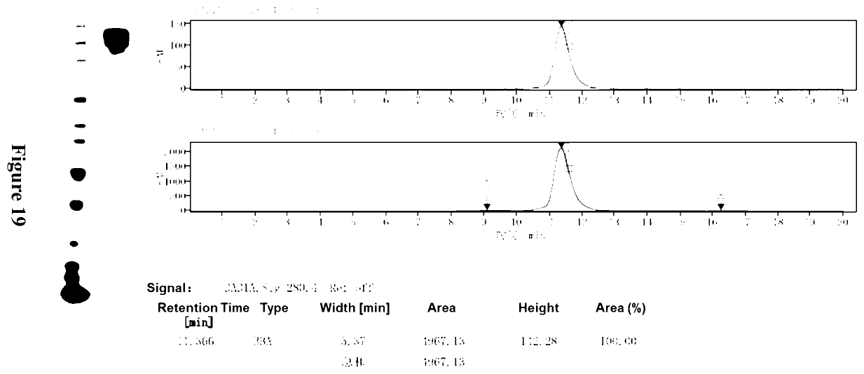

For ACF_389 and ESB07.451, to get purer sample, Mono S chromatography (5/50 GL, Cytiva) was utilized. Antibodies were loaded and washed with buffer A (10 mM sodium acetate, pH 5.2), then eluted with a buffer B gradient (10 mM sodium acetate, pH 5.2, 250 mM NaCl). The results for ACF_389 are shown in FIGS. 18 and 19. The results for ESB07.451 are shown in FIGS. 22 and 23.

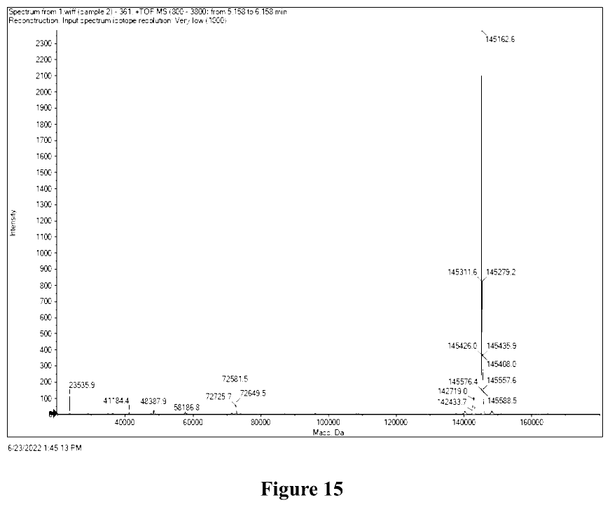

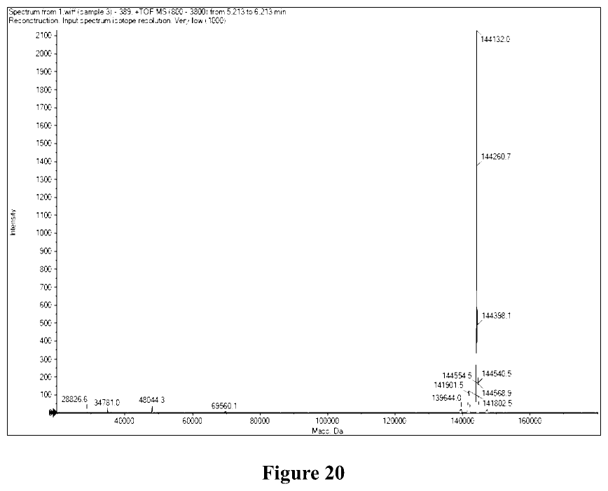

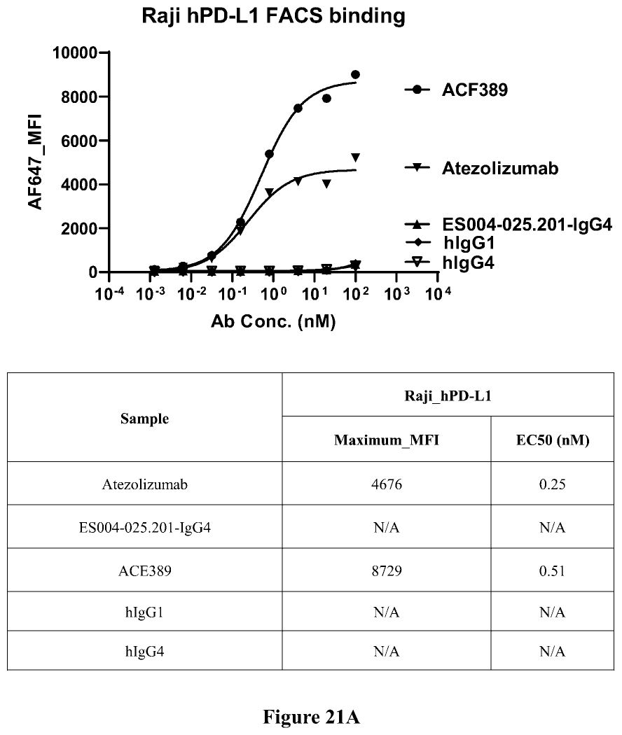

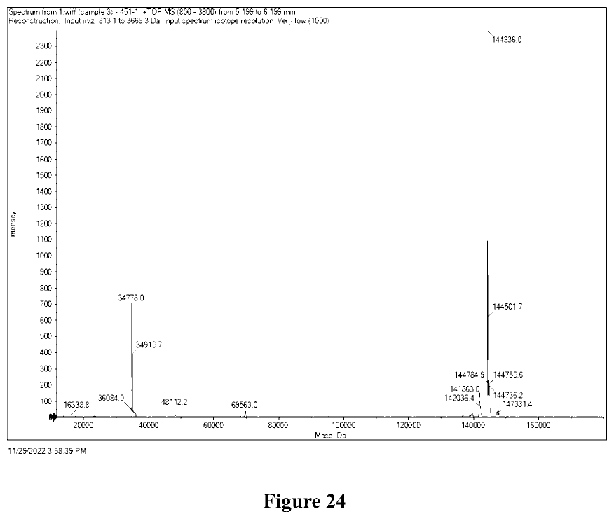

In order to quantitatively evaluate the efficiency of cognate CH1 and CL chain pairing, the purified antibodies were analyzed by LC-MS under non-reducing condition. The results for ACF_361, ACF_389 and ESB07.451 are shown in FIG. 15, FIG. 20 and FIG. 24, respectively. From the LC-MS results, the peak around 145KD was the cognate CH1/CL pairing which showed the three antibodies were correctly assembled.

### 5.2 Binding Affinity Assay

5.2.1 Binding Affinity with CLDN18.2 and SIRPα Overexpressing Cells

The dual binding property of the bispecific antibody ACF_361 to the cells were analyzed by FACS.

1×10⁵ CLDN18.2 expressing MC38 cells per well were incubated with each of the purified antibodies from 100 nM to 0.05 nM for 1 hr and then incubated with 2nd antibody Alexa Flour 647 anti-human IgG (H+L).

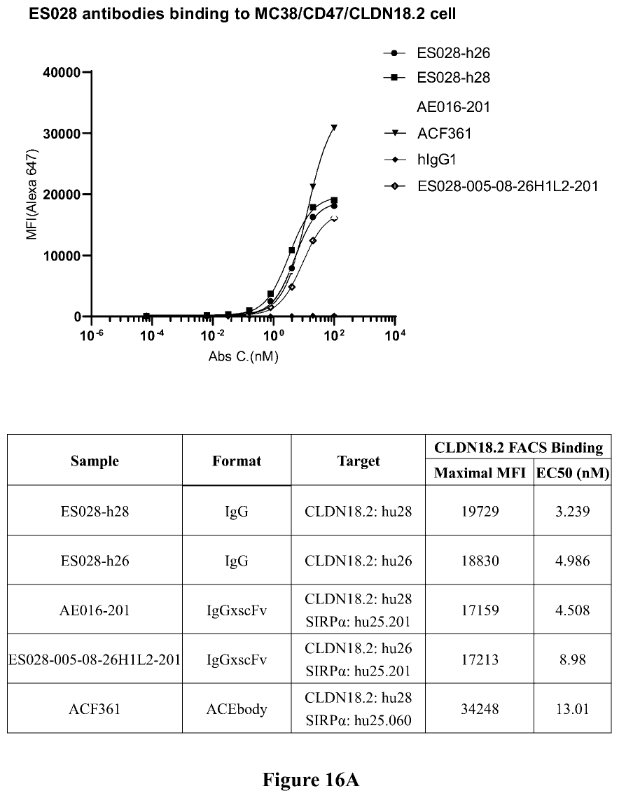

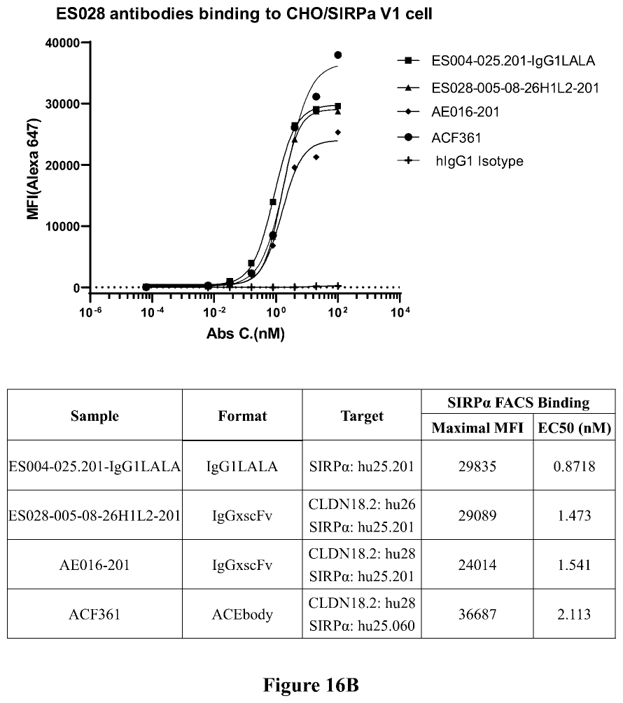

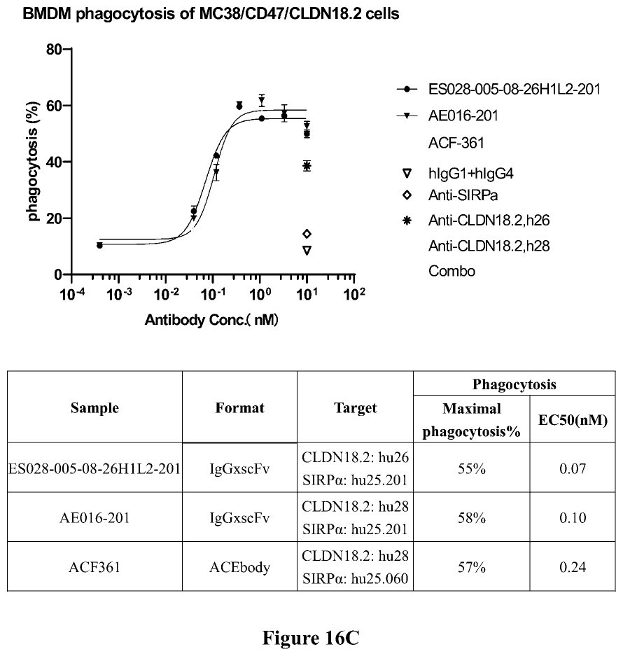

In this assay, ES028-h26, ES028-h28, AE016-201, and ES028-005-08-26H1L2-201 were used as positive controls, and hIgG1 (an isotype control which does not specifically bind to CLDN18.2 or SIRPα, the same below) was used as the negative control. Information for each tested antibody is shown in FIG. 16A. Both ES028-h26 and ES028-h28 were mono-specific IgG antibodies targeting CLDN18.2. AE016-201 was a bi-specific antibody (in which an scFv was linked to an IgG) targeting CLDN18.2 and SIRPα. ES028-005-08-26H1L2-201 was a bi-specific antibody (in which an scFv was linked to an IgG) targeting CLDN18.2 and SIRPα.

The binding curve of the antibodies to MC38-CLDN18.2 cells are shown in FIG. 16A.

1×10⁵ CHO/SIRPαV1 cells per well were incubated with each of the purified antibodies from 100 Nm to 0.05 Nm for 1 hr and then incubated with 2^{nd} antibody Alexa Flour 647 anti-human IgG (H+L).

In this assay, ES001-025.201-IgG1LALA, ES028-005-08-26H1L2-201, AE016-201 were used as positive controls, and hIgG1 Isotype was used as the negative control. Information for each tested antibody is shown in FIG. 16B. ES001-025.201-IgG1LALA was a mono-specific IgG antibody targeting SIRPα with with LALA mutations on the Fc region of the antibody. Both ES028-005-08-26H1L2-201 and AE016-201 were bi-specific antibodies (in which an scFv was linked to an IgG) targeting CLDN18.2 and SIRPα.

The binding curve of the antibodies to CHO/SIRPαV1 cell are shown in FIG. 16B.

5.2.2 Binding Affinity with PDL1 and SIRPα Overexpressing Cells

The dual binding property of the bispecific antibody ACF_389 to the cells were analyzed by FACS. 1×10⁵ PDL1 expressing Raji cells per well were incubated with each of the purified antibodies from 100 nM to 0.05 nM for Ihr and then incubated with 2nd antibody Alexa Flour 647 anti-human IgG (H+L).

In this assay, Atezolizumab was used as a positive control, and ES004-025.201-IgG4, hIgG1 and hIgG4 (an isotype control which does not specifically bind to CLDN18.2 or SIRPα, the same below) were used as the negative controls. Atezolizumab is an known anti-PDL1 antibody, whose detailed information can be found in, for example, U.S. Pat. No. 9,873,740B2. ES004-025.201-IgG4 was a mono-specific antibody targeting SIRPα.

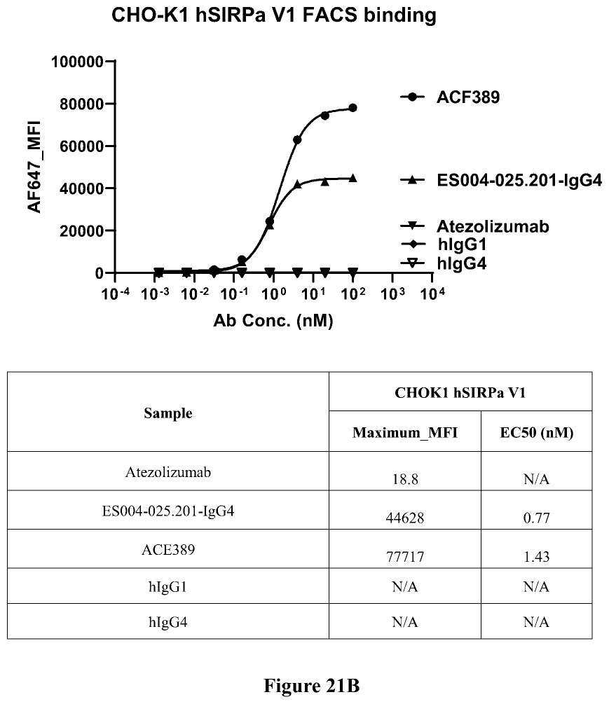

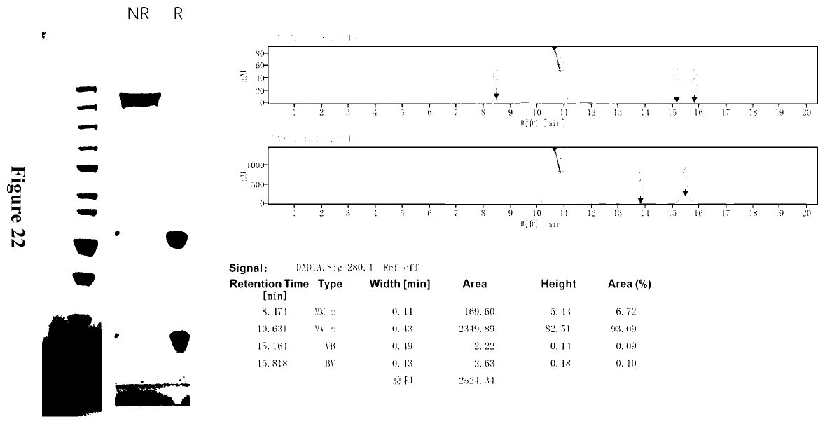

The binding curve of the antibodies to Raji/PDL1 cells are shown in FIG. 21A.

1×10⁵ CHO/SIRP V1 cell per well were incubated with each of the purified antibodies from 100 nM to 0.05 nM for 1 hr and then incubated with 2nd antibody Alexa Flour 647 anti-human IgG (H+L).

In this assay, ES004-025.201-IgG4 was used as a positive control, and Atezolizumab, hIgG1 and hIgG4 were used as the negative controls. Information of the antibodies are described above.

The binding curve of the antibodies to CHO/SIRPαV1 cell are shown in FIG. 21B.

### 5.3 Phagocytosis Activity of Bispecific Antibody

Phagocytosis activity of ACF_361 was tested in this assay.

To get single cell suspension, microphage cells were digested with accutase at 37° C. for 10 mins, then cell culture medium was added to stop the digestion. After centrifugation at 400 g, 5 mins, macrophage cells were replated to 96-well plate at 3×10⁴/well and recovered at 37° C. overnight. Mouse BMDM cells were stained with CD11b and F4/80 to evaluate the quality and purity. The next day, 25 ul diluted Abs with concentration from 0.05 nM to 10 nM and 50 ul 6×10⁴ Violet CSFE labeled Raji-CLDN18.2 cells per well were added and incubated at 37° C. for 3 hrs. Cells were washed with DPBS, and digested with accutase at 37° C. for 10 mins, and then digested cells were washed with FACS buffer. Cells were incubated with Anti mouse F4/80 APC at 4° C. for 30 mins. The stained cells were washed and suspended with FACS buffer. The percentage of phagocytosis was determined by FACS.

In this assay, ES028-005-08-26H1L2-201 and AE016-201 were used as positive controls, and hIgG1+hIgG4, an anti-SIRPα mono-specific antibody, two anti-CLDN18.2 antibodies (Anti-CLDN18.2.h26 and Anti-CLDN18.2.h28) and combo were used as negative controls. Both ES028-005-08-26H1L2-201 and AE016-201 were bi-specific antibodies (in which an scFv was linked to an IgG) targeting CLDN18.2 and SIRPα. Equal molar of anti-CLDN18.2 hu26 (hIgG1) and anti-SIRPα hu25.060 (hIgG4) were added as combo treatment group (i.e., combo). Equal molar of hIgG1 and hIgG4 were added as isotype control group (i.e., hIgG1+hIgG4).

Summarized data was shown in FIG. 16C.

### 5.4 Hydrophobicity Evaluation

Retention time in HIC-HPLC assay is a factor for hydrophobicity evaluation of an antibody. 15 ul of 0.25 mg/ml antibody was injected into MacPac-10 HIC column in Agilent 1260 system and signal at 280 nm/214 nm was detected. As shown in Table 13, retention time of ACF_361, ACF_389 and ESB07.451 in MacPac-10 HIC is 8.413 min, 13.583 min, and 9.9 min, respectively, which indicates low hydrophobicity of the three bispecific antibodies.

TABLE 13
Retention time of antibodies in HIC-HPLC assay

| Sample Name | RT-HIC-UPLC (min) |
|---|---|
| ACF_361 | 8.413 |
| ACF_389 | 13.583 |
| ESB07.451 | 9.9 |

### 5.5 Stability Test

### 5.5.1 Thermostability Assessment

The melting temperature (Tm) values of bispecific antibody in PBS buffer was detected to predict its thermostability. Briefly, the bispecific antibody was resolved in 10 mM PBS buffer (pH7.2-7.4). The Tm values were then detected by Differential Scanning Fluorimetry (DSF) using QuantStudio 7 Flex Real time PCR system. As shown in Table 14, Tm value of the ACF_361, ACF_389 and ESB07.451 indicates that the bispecific antibody has a good thermostability.

TABLE 14
Tm Values of Antibodies

| Sample Name | Tm1-DSF (° C.) | Tm2-DSF (° C.) |
|---|---|---|
| ACF_361 | 68.7 | 80.7 |
| ACF_389 | 68.7 | 84.8 |
| ESB07.451 | 69.2 | 74.3 |

### 5.5.2 Low pH Stability Assessment

Good stability under low pH conditions is important for antibodies because the purification process often involves exposure to acidic solution conditions and the commonly used low pH virus inactivation. Low pH usually causes soluble, insoluble aggregates and accelerates fragmentations. Therefore, to assess protein stability under low pH condition, the bispecific antibody in PBS buffer were titrated with acetic acid to pH 3.0-3.5. The antibody stability was then assessed through monitoring the changes in total soluble protein (recovery) and purity before and after exposure to the low pH solution for 2 hours.

The results are shown in Table 15, wherein TO indicates the data of the original samples and low pH 2 h indicates the data in low pH after 2 hours. It can be seen that 2 hours exposure to the low pH solution resulted in nearly no change in total purity, indicating that all of the three antibodies ACF_361, ACF_389 and ESB07.451 have a good stability under low pH conditions.

TABLE 15
Low pH Stability of Antibodies

| | | SEC-280 nm | | |
|---|---|---|---|---|
| Sample Name | Condition | Aggregate % | Purity % | Fragment % |
| ACF_361 | T0 | 0.8 | 98.7 | 0.5 |
| | Low pH 2 h | 0.8 | 98.8 | 0.4 |
| ACF_389 | T0 | 0.4 | 99.7 | ND |
| | Low pH 2 h | 0.6 | 99.4 | ND |
| ESB07.451 | T0 | 2.1 | 97.9 | ND |
| | low pH 2 h | 2.2 | 97.8 | ND |

### 5.5.3 Stability Under Freeze-Thaw Stress

Freeze-thaw stability is often explored to determine the susceptibility of antibodies to temperature cycling which products are frequently exposed. For example, drug substance is often frozen to enable long-term storage. Drug product may be exposed to frozen temperature as part of the lyophilization process. The major degradation pathway of freeze-thaw is aggregates, including precipitates, particles, and soluble particles. Therefore, freeze-thaw stability of the bispecific antibody was assessed through monitoring the changes in purity before and after repeated freeze-thaw treatments. Briefly, antibody in 20 mM PBS (pH6.0-6.2) was frozen to −80° C. for more than 12 hours and then thawed at room temperature. After 5 repeated freeze-thaw cycles, the stress-treated sample was assayed for concentration and purity.

The results are shown in Table 16, wherein TO indicates the data of the original samples and FT5 indicates the data after freeze and thaw for five times. It can be seen that none of ACF_361, ACF_389 and ESB07.451 had obvious aggregates and fragments increase after multiple freeze-thaw cycles, indicating that all of the three antibodies have a good freeze-thaw stability.

TABLE 16
Stability under Freeze-thaw Stress of Antibodies

| | | SEC-280 nm | | |
|---|---|---|---|---|
| Sample name | Condition | Aggregate % | Purity % | Fragment % |
| ACF_361 | T0 | 0.8 | 98.7 | 0.5 |
| | FT5 | 1.2 | 98.4 | 0.4 |
| ACF_389 | T0 | 0.4 | 99.7 | ND |
| | FT5 | 0.5 | 99.5 | ND |
| ESB07.451 | T0 | 2.1 | 97.9 | ND |
| | FT5 | 2.4 | 97.7 | ND |

### 5.5.4 Stability Under Thermal Stress

Thermal stress at temperatures exceeding normal storage conditions can accelerate degradation, thereby increasing the detectability of potential degradation pathways to provide information about long-term degradation at intended storage conditions. The thermal stress test of the bispecific antibody in PBS was performed at 25° C. and/or 40° C. 4 weeks. SEC-purity was detected to monitor insoluble or soluble aggregates.

The results are shown in Table 17, wherein TO indicates the data of the original samples, 25C-2W indicates the data under 25° C. for 2W and other naming rules are similar. It can be seen that purity of ACF_361 and ACF_389 in PBS changed little after 4 weeks incubation at 25° C., and purity of ESB07.451 in PBS changed little after 4 weeks of incubation at 25° C. and 40° C.

TABLE 17
Stability under Thermal Stress of Antibodies

| | | SEC-280 nm | | | CE-NR | | | |
|---|---|---|---|---|---|---|---|---|
| Sample | | Aggregate | Purity | Fragment | Main | CE-R | | |
| Name | Condition | % | % | % | Peak % | LC % | HC % | Purity % |
| ACF_361 | T0 | 0.8 | 98.7 | 0.5 | 90.0 | 32.2 | 63.6 | 95.8 |
| | 25 C.-2 W | 0.9 | 98.5 | 0.6 | NA | NA | NA | NA |
| | 25 C.-4 W | 1.2 | 98.0 | 0.8 | NA | NA | NA | NA |
| ACF_389 | T0 | 0.4 | 99.7 | ND | 98.6 | 31.7 | 67.8 | 99.5 |
| | 25 C.-2 W | 0.8 | 97.9 | 1.3 | NA | NA | NA | NA |
| | 25 C.-4 W | 1.0 | 97.6 | 1.4 | NA | NA | NA | NA |
| ESB07.451 | T0 | 2.1 | 97.9 | ND | NA | NA | NA | NA |
| | 25 C.-3 W | 2.2 | 97.8 | ND | NA | NA | NA | NA |
| | 25 C.-4 W | 2.3 | 97.8 | ND | NA | NA | NA | NA |
| | 40 C.-3 W | 2.2 | 97.2 | 0.6 | NA | NA | NA | NA |
| | 40 C.-4 W | 2.2 | 97.0 | 0.8 | NA | NA | NA | NA |

1. An antibody or antigen-binding fragment thereof, which comprises a first arm formed by a first heavy chain and a first light chain with an interchain disulfide bond formed therebetween, and a second arm formed by a second heavy chain and a second light chain with an interchain disulfide bond formed therebetween,
wherein at least one non-cysteine residue on the first heavy chain is substituted with cysteine, wherein the non-cysteine residue is at a position selected from the group consisting of amino acid positions 126, 128, 129, 136, 141, 168, 170, 173, 175 or 187 of the first heavy chain; and
at least one non-cysteine residue on the first light chain is substituted with cysteine, wherein the non-cysteine residue is at a position selected from the group consisting of amino acid positions 114, 116, 118, 124, 135, 137, 138, 160, 162 or 164 of the first light chain.

2. The antibody or antigen-binding fragments thereof of claim 1, wherein
(I) the at least one non-cysteine residue on the first heavy chain substituted with cysteine is selected from the group consisting of amino acid residues F126, L128, A129, S136, A141, H168, F170, V173, Q175 or T187 of the first heavy chain; and at least one non-cysteine residue on the first light chain substituted with cysteine is selected from the group consisting of amino acid residues S114, F116, F118, Q124, L135, N137, N138, Q160, S162 or T164 of the first light chain; and/or
(II) the interchain disulfide bond between the second heavy chain and the second light chain is formed between a native cysteine on the second light chain and a native cysteine on the second heavy chain; wherein the position of the native cysteine on the second light chain is corresponding to position C214 on the human kappa chain, and the position of the native cysteine on the second heavy chain is corresponding to position C220 on the human IgG1; and/or
(III) the first arm and the second arm can specifically bind to an antigen selected from the group consisting of: GPC3, SIRPα, CLDN18.2, Siglec15, HER2 EGFR, CD19, CD20, CD39, CD47, PG1, PDL1, CD3, NKG2D, NKG2A, Nkp46, CD137, OX40, CD40, LILRB1, LILRB2, LILRB4, TROP2, CD112, TIGIT, FAP, VEGFA, DLL4, ANG-2, wherein the first arm and the second arm specifically bind to different antigens.

3. The antibody or antigen-binding fragments thereof of claim 1, wherein the first light chain and the first heavy chain comprises at least one non-cysteine substitution pair of a native cysteine on the first light chain and a native cysteine on the first heavy chain, and at least one cysteine substitution pair selected from the group consisting of:
(i) S114C on light chain and S136C on heavy chain;
(ii) F116C on light chain and S136C on heavy chain;
(iii) F118C on light chain and L128C on heavy chain;
(iv) F118C on light chain and A129C on heavy chain;
(v) F118C on light chain and A141C on heavy chain;
(vi) Q124C on light chain and F126C on heavy chain;
(vii) L135C on light chain and F170C on heavy chain;
(viii) N137C on light chain and F170C on heavy chain;
(ix) N138C on light chain and H168C on heavy chain;
(x) Q160C on light chain and V173C on heavy chain;
(xi) Q160C on light chain and Q175C on heavy chain;
(xii) S162C on light chain and F170C on heavy chain;
(xiii) S162C on the light chain and T187C on heavy chain; and
(xiv) T164C on light chain and F170C on heavy chain;
wherein the at least one cysteine substitution pair forms at least one interchain disulfide bond between the first heavy chain and the first light chain;
wherein the native cysteines on the first heavy chain and the first light chain are independently substituted with any one of non-cysteine residues.

4. The antibody or antigen-binding fragments thereof of claim 3, the native cysteines on the first heavy chain and the first light chain are independently substituted with any one of Serine, Alaine, Glycine or Valine.

5. The antibody or antigen-binding fragment thereof of claim 1,
wherein;
(1) the first heavy chain and the first light chain comprises one cysteine substitution pair; or
(2) the first heavy chain and the first light chain comprises two cysteine substitution pairs.

6. (canceled)

7. The antibody or antigen-binding fragment thereof of claim 5, wherein the two cysteine substitution pairs are selected from the group consisting of:
(i) S114C/Q160C on light chain and S136C/V173C on heavy chain;
(ii) F118C/Q124C on light chain and L128C/F126C on heavy chain;
(iii) F118C/Q124C on light chain and A129C/F126C on heavy chain;
(iv) F118C/Q124C on light chain and A141C/F126C on heavy chain;
(v) F116C/Q124C on light chain and S136C/F126C on heavy chain;
(vi) Q124C/T164C on light chain and F126C/F170C on heavy chain;
(vii) Q124C/N138C on light chain and F126C/H168C on heavy chain;
(viii) Q160C/L135C on light chain and Q175C/F170C on heavy chain;
(ix) S114C/S162C on light chain and S136C/F170C on heavy chain;
(x) F118C/S162C on light chain and L128C/F170C on heavy chain;
(xi) F118C/S162C on light chain and A141C/F170C on heavy chain;
(xii) Q124C/S162C on light chain and F126C/F170C on heavy chain;
(xiii) N137C/S162C on light chain and F170C/T187C on heavy chain;
(xiv) Q124C/T164C/C214S on light chain and F126C/F170C/C220S on heavy chain; and
(xv) Q124C/S162C/C214S on light chain and F126C/F170C/C220S on heavy chain.

8. (canceled)

9. The antibody or antigen-binding fragment thereof of claim 1, wherein the second light chain and the second heavy chain comprises at least one non-cysteine substitution pair of a native cysteine substituted to Serine on the light chain and a native cysteine substituted to Serine on the heavy chain, and at least one cysteine substitution pair selected from the group consisting of:
(i) S114C on light chain and S136C on heavy chain;
(ii) F116C on light chain and S136C on heavy chain;
(iii) F118C on light chain and L128C on heavy chain;
(iv) F118C on light chain and A129C on heavy chain;
(v) F118C on light chain and A141C on heavy chain;
(vi) Q124C on light chain and F126C on heavy chain;
(vii) L135C on light chain and F170C on heavy chain;
(viii) N137C on light chain and F170C on heavy chain;
(ix) N138C on light chain and H168C on heavy chain;
(x) Q160C on light chain and V173C on heavy chain;
(xi) Q160C on light chain and Q175C on heavy chain;
(xii) S162C on light chain and F170C on heavy chain;
(xiii) S162C on the light chain and T187C on heavy chain; and
(xiv) T164C on light chain and F170C on heavy chain;
wherein the at least one cysteine substitution pair forms at least one interchain disulfide bond between the second light chain and the second heavy chain, and the cysteine substitution pair(s) on the first arm is different from the cysteine substitution pair(s) on the second arm.

10. The antibody or antigen-binding fragment thereof of claim 1,
wherein:
(i) the native cysteine substituted on the second light chain is at position C214 on the human kappa chain, and the native cysteine substituted on the second heavy chain is at position C220 on the human IgG1; and/or
(ii) the second heavy chain and the second light chain comprises two cysteine substitution pairs.

11. (canceled)

12. The antibody or antigen-binding fragment thereof of claim 10, wherein the two cysteine substitution pairs are selected from the group consisting of:
(i) S114C/Q160C on light chain and S136C/V173C on heavy chain;
(ii) F118C/Q124C on light chain and L128C/F126C on heavy chain;
(iii) F118C/Q124C on light chain and A129C/F126C on heavy chain;
(iv) F118C/Q124C on light chain and A141C/F126C on heavy chain;
(v) F116C/Q124C on light chain and S136C/F126C on heavy chain;
(vi) Q124C/T164C on light chain and F126C/F170C on heavy chain;
(vii) Q124C/N138C on light chain and F126C/H168C on heavy chain;
(viii) Q160C/L135C on light chain and Q175C/F170C on heavy chain;
(ix) S114C/S162C on light chain and S136C/F170C on heavy chain;
(x) F118C/S162C on light chain and L128C/F170C on heavy chain;
(xi) F118C/S162C on light chain and A141C/F170C on heavy chain;
(xii) Q124C/S162C on light chain and F126C/F170C on heavy chain; and
(xiii) N137C/S162C on light chain and F170C/T187C on heavy chain;
wherein the cysteine substitution pair(s) on the first arm is different from the cysteine substitution pairs on the second arm.

13. The antibody or antigen-binding fragment thereof of claim 1, wherein
the first heavy chain constant region and/or the second heavy chain constant region comprises a human IgG1, IgG2, IgG3 or IgG4; and
the first light chain constant region and/or the second light chain constant region comprises a human kappa light chain or a human lambda light chain.

14. The antibody or antigen-binding fragment thereof of claim 1, wherein the first heavy chain and the second heavy chain form a heterodimer; and the Fc region of the first heavy chain constant region and/or the Fc region of the second heavy chain constant region comprises one or more modifications facilitating the heterodimerization.

15. The antibody or antigen-binding fragment thereof of claim 14, wherein the antibody or an antigen-binding fragment thereof comprises one or more features selected from the group consisting of:
(a) the Fc region of the first heavy chain interacts with the Fc region of the second heavy chain through a Knob/Hole structure;
(b) the modifications facilitating the heterodimerization comprise introduction of cysteine residues capable of forming an interchain disulfide bond;
(c) the Fc region of the first heavy chain constant region and the Fc region of the second heavy chain constant region comprise modifications in the CH3 regions, respectively wherein the modifications in the two CH3 regions are selected from the following:

| Modification(s) in one | Modification(s) in the other |
|---|---|
| CH3 region | CH3 region |
| T366S/L368A/Y407V | T366W |
| S354C/T366W | Y349C/T366S/L368A/Y407V |
| T366Y | Y407T |
| T366W | Y407A |
| T394W | F405A |
| T366Y/F405A | T394W/Y407T |
| T366W/F405W | T394S/Y407A |
| F405W | T394S |
| D399C | K392C |
| T366W/D399C | T366S/L368A/K392C/Y407V |
| T366W/K392C | T366S/L368A/D399C/Y407V |
| S354C/T366W | Y349C/T366S/L368A/Y407V |
| Y349C/T366W | S354C/T366S/L368A/Y407V |
| E356C/T366W | Y349C/T366S/L368A/Y407V |
| Y349C/T366W | E356C/T366S/L368A/Y407V |
| E357C/T366W | Y349C/T366S/L368A/Y407V |
| Y349C/T366W | E357C/T366S/L368A/Y407V |

(d) the Fc region of the first heavy chain constant region and the Fc region of the second heavy chain constant region comprise modifications in the CH3 regions, respectively; wherein the modifications in the two CH3 regions are selected from the following:

| Modification(s) in one | Modification(s) in the other |
|---|---|
| CH3 region | CH3 region |
| K370E/D399K/K439D | D356K/E357K/K409D |
| K409D | D399K |
| K409E | D399K |
| K409E | D399R |
| K409D | D399R |
| D339K | E356K |
| E356K/D399K | K392D/K409D |
| E356K/D399K | K409D/K439D |
| E357K/D399K | K370D/K409D |
| E356K/E357K/D399K | K370D/K392D/K409D |
| E357K/D399K | K392D/K409D |
| K392D/K409D | D399K |
| K360D/L409D | D399K |

and
(e) the Fc region of the first heavy chain constant region and/or the Fc region of the second heavy chain constant region further comprises modifications improving the stability of the antibody or the antigen-binding fragment.

16. The antibody or antigen-binding fragment thereof of claim 15, wherein the Fc region of the first heavy chain comprises the Knob mutations, and the Fc region of the second heavy chain comprises the Hole mutations; or the Fc region of the first heavy chain comprises the Hole mutations, and the Fc region of the second heavy chain comprises the Knob mutations.

17. The antibody or antigen-binding fragment thereof of claim 16, wherein the Knob mutations comprise T366W, and the Hole mutations comprise T366S/L368A/Y407V.

18-22. (canceled)

23. The antibody or an antigen-binding fragment thereof of claim 1, wherein the antibody or an antigen-binding fragment thereof comprises one or more features selected from the group consisting of:
(a) the antibody or an antigen-binding fragment thereof is humanized of chimeric antibody;
(b) the antibody or an antigen-binding fragment thereof is a bispecific antibody or a multi-specific antibody; and
(c) the antibody or an antigen-binding fragment thereof is linked to one or more conjugate moieties.

24-25. (canceled)

26. The antibody or an antigen-binding fragment thereof of claim 23, wherein the conjugate moiety comprises a second antibody fragment.

27. The antibody or an antigen-binding fragment thereof of claim 26, wherein the antibody or antigen-binding fragment thereof comprises an Fab fragment, which is linked to the C-terminus of the Fc region(s) of the second antibody fragment, and/or
the conjugate moiety comprises an agent for detection or isolation, such as clearance-modifying agent, a luminescent label, a fluorescent label, an enzyme-substrate label, or a purification moiety, and/or
the conjugate moiety comprises a therapeutic agent or a drug.

28-29. (canceled)

30. An isolated polynucleotide encoding the antibody or an antigen-binding fragment thereof of claim 1.

31. A vector comprising the isolated polynucleotide of claim 30.

32. A host cell comprising the vector of claim 31.

33. A pharmaceutical composition, comprising:
(i) the antibody or an antigen-binding fragment thereof of claim 1, and
(ii) one or more pharmaceutically acceptable carriers.

34. A method of expressing the antibody or an antigen-binding fragment thereof of claim 1.

35. A method of treating, preventing or alleviating a disease in a subject, comprising administering to the subject a therapeutically effective amount of the antibody or an antigen-binding fragment thereof of claim 1.

36. The method of claim 35, wherein the subject is human.

37-38. (canceled)


SEQUENCE LISTING (21 sequences)

SEQ ID NO: 1 - protein, 112 aa, synthetic construct
  REGION 1..112: note = Synthetic
EVQVVESGGGLVQSGGSLRLSCAGSGFTESAGFMVWHRQVPGKERELVALIATPSGSTQYADSVKGRFTISRDNGKNTVYLQMNSLKPEDTAVYYCNIRGYWGQGTLVTVSS

SEQ ID NO: 2 - protein, 113 aa, synthetic construct
  REGION 1..113: note = Synthetic
DIVMTQSPDSLAVSLGERATINCKSSQSLLNAGNQKNYLTWYQQKPGQPPKLLIYWSSTRESGVPDRFSGSGSGTDFTLTISSLQAEDVAVYHCQNNYYYPLTFGGGTKLEIK

SEQ ID NO: 3 - protein, 117 aa, synthetic construct
  REGION 1..117: note = Synthetic
QVQLVQSGAEVKKPGASVKVSCKASGYTFTNWVHWVRQAPGQGLEWMGEINPTNARSNYNEKFKKRVTMTRDTSTSTVYMELSSLRSEDTAVYYCARIYYGNSFAHWGQGTLVTVSS

SEQ ID NO: 4 - protein, 107 aa, synthetic construct [sequence omitted]

SEQ ID NO: 5 - protein, 103 aa, synthetic construct
  REGION 1..103: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 6 - protein, 227 aa, synthetic construct
  REGION 1..227: note = Synthetic
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 227 aa, synthetic construct
  REGION 1..227: note = Synthetic
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 8 - protein, 227 aa, synthetic construct [sequence omitted]

SEQ ID NO: 9 - protein, 5 aa, synthetic construct
  REGION 1..5: note = Synthetic
GGGGS

SEQ ID NO: 10 - protein, 113 aa, synthetic construct
  REGION 1..113: note = Synthetic
EVQLVQSGAEVKKPGATVKISCKVSGFNIKDYYMHWVRQAPGKGLEWIGRVDPEDAETKYAEKFQGRVTITADTSTDTAYMELSSLRSEDTAVYYCNRGLVYWGQGTLVTVSS

SEQ ID NO: 11 - protein, 108 aa, synthetic construct
  REGION 1..108: note = Synthetic
EIVLTQSPATLSLSPGERATLSCRASQSVSSSYLYWYQQKPGQAPKLLIYSASNRASGIPARFSGSGSGTDYTLTISSLEPEDFAVYYCHQWSSYPYTFGQGTKLEIK

SEQ ID NO: 12 - protein, 118 aa, synthetic construct [sequence omitted]

SEQ ID NO: 13 - protein, 107 aa, synthetic construct [sequence omitted]

SEQ ID NO: 14 - protein, 117 aa, synthetic construct
  REGION 1..117: note = Synthetic
QVQLVQSGAEVKKPGASVKVSCKASGYTFTDYYINWVRQAPGQRLEWMGWIYPGSGNTKYNEKFKGRVTITRDTSASTAYMELSSLRSEDTAVYYCARGLLGPGDYWGQGTTVTVSS

SEQ ID NO: 15 - protein, 107 aa, synthetic construct
  REGION 1..107: note = Synthetic
DIVMTQSPDSLAVSLGERATINCKASQSVSNDVAWYQQKPGQPPKLLIYYASNRYTGVPDRFSGSGSGTDFTLTISSLQAEDVAVYYCQQDYSSPYTFGGGTKLEIK

SEQ ID NO: 16 - protein, 116 aa, synthetic construct
  REGION 1..116: note = Synthetic
QVQLVQSGAEVKKPGASVKVSCKASGYTFTSYWMYWVRQAPGQGLEWIGHINPSSGYTKYNQKFKDKATLTADKSTSTAYMELSSLRSEDTAVYYCARDPNWDNYWGQGTTVTVSS

SEQ ID NO: 17 - protein, 107 aa, synthetic construct
  REGION 1..107: note = Synthetic
DIQMTQSPSSMSASVGDRVTITCKASQDVSIAVAWYQQKPGKAPKLLIYSASYRYTGVPSRFSGSGSGTDFTFTISSVQPEDIATYYCQQHYSTPYTFGGGTKLEIK

SEQ ID NO: 18 - protein, 103 aa, synthetic construct
  REGION 1..103: note = Synthetic
ASTKGPSVCPLAPCSRSTSESTAALGCLVKDYFPEPVTVSWNSGALTSGVHTCPAVLQSSGLYSLSSVVTVPSSSLGTKTYTCNVDHKPSNTKVDKKVEPKSC

SEQ ID NO: 19 - protein, 107 aa, synthetic construct
  REGION 1..107: note = Synthetic
RTVAAPSVFIFPPSDECLKSGTASVVCLLNNFYPREAKVQWKVDNALQSGNSQECVTEQDSKDSTYSLSSTLTLSKADYEKHKVYACEVTHQGLSSPVTKSFNRGES

SEQ ID NO: 20 - protein, 227 aa, synthetic construct [sequence omitted]

SEQ ID NO: 21 - protein, 227 aa, synthetic construct
  REGION 1..227: note = Synthetic
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK